(19) United States
(12) Patent Application Publication
(10) Pub. No.: US 2004/0030106 A1
(43) Pub. Date: Feb. 12, 2004

(54) Novel compounds and process

(76) Inventors: Martin Friede, Rixensart (BE); Sean Mason, Cambridge (GB); William Gordon Turnell, Cambridge (GB); Carlota Vinals Y De Bassols, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/362,527

(22) PCT Filed: Aug. 17, 2001
(86) PCT No.: PCT/EP01/09576

(30) Foreign Application Priority Data
Aug. 22, 2000 (GB) 00207717.5

(51) Int. Cl. A61K 39/00; C07K 14/47

(52) U.S. Cl. 530/404; 424/185.1

(57) ABSTRACT

The present invention relates to a novel chemical process for the covalent conjugation of disulphide bridge cyclised peptides to immunogenic carrier molecules by thio-ether linkages to form vaccine immunogens. In particular, the novel chemistry involves reacting a thiolated carrier with a cyclic peptide containing a disulphide bridge, which cylcic peptide (herein a disulphide bridge cyclised peptide) has attached to it, usually via a linker, a reactive group capable for forming thio-ether bonds with the carrier. The invention further related to activated peptide intermediates of the process, medicaments produced by the process, pharmaceutical compositions containing the medicaments, and the use of the pharmaceutical compositions in medicine. The process of the present invention is particularly useful for the preparation of highly pure immunogens for vaccines, comprising disulphide bridge cyclised peptides. Also novel immunogens are provided, base don peptides derived from the sequence of human IgE, which are useful in the immunotherapy of allergy. Accordingly, the inventions related also to a process for conjugation of IgE disulphide bridge cyclised peptides to carrier, immunogens produced by the process and vaccines and pharmaceutical compositions comprising them and their use in the treatment of allergy.

The present invention relates to a novel chemical process for the covalent conjugation of disulphide bridge cyclised peptides to immunogenic carrier molecules by thio-ether linkages to form vaccine immunogens. In particular, the novel chemistry involves reacting a thiolated carrier with a cyclic peptide containing a disulphide bridge, which cyclic peptide (herein a disulphide bridge cyclised peptide) has attached to it, usually via a linker, a reactive group capable of forming thio-ether bonds with the carrier. The invention further relates to activated peptide intermediates of the process, medicaments produced by the process, pharmaceutical compositions containing the medicaments, and the use of the pharmaceutical compositions in medicine. The process of the present invention is particularly useful for the preparation of highly pure immunogens for vaccines, comprising disulphide bridge cyclised peptides. Also novel immnunogens are provided, based on peptides derived from the sequence of human IgE, which are useful in the immunotherapy of allergy. Accordingly, the invention relates also to a process for conjugation of IgE disulphide bridge cyclised peptides to carriers, immunogens produced by the process and vaccines and pharmaceutical compositions comprising them and their use in the treatment of allergy.

Immunogens comprising short peptides are becoming increasingly common in the field of vaccine prophylaxis or therapy. In many disease states it is often possible, and desirable, to design vaccines comprising a short peptide rather than a large protein. Peptides which may be used as immunogens may be the full length native protein, for example human peptidic hormones, or may be fragments of a larger antigen derived from a given pathogen, or from a large self-protein. For example, short peptides of IgE may be used for prophylaxis of allergy, whereas the use of IgE itself as the immunogen may induce anaphylactic shock.

It has previously been thought that amongst the problems associated with the peptide approach to vaccination, is the fact that peptides per se are poor immunogens. Generally the sequences of the peptides chosen are such that they include a B-cell epitope to provide a target for the generation of anti-peptide antibody responses, but because of their limited size rarely encompass sufficient T-cell epitopes in order to provide the necessary cytokine help in the induction of strong immune responses following priming and boosting applications of the vaccine.

Strategies to overcome this problem of immunogenicity include the linking of the peptide to large highly immunogenic protein carriers. The carrier proteins contain a large number of peptidic T-cell epitopes which are capable of being loaded into MHC molecules, thereby providing bystander T-cell help, and/or alternatively the use of strong adjuvants in the vaccine formulation. Examples of these highly immunogenic carriers which are currently commonly used for the production of peptide immunogens include the Diptheria and Tetanus toxoids (DT and TT respectively), Keyhole Limpet Haemocyanin (KLH), and the purified protein derivative of Tuberculin (PPD).

Peptides used in a particular vaccine immunogen are often chosen such that they generate an antibody response to the location site of that peptide in the context of the full length native protein. Thus, in order to generate antibodies that bind to such chosen locations, the peptide in the immunogen must assume substantially the same shape as it would exist if it was confined by the flanking regions of the full length native protein. However, merely conjugating a linear peptide sequence, by conventional chemistry, to a carrier protein rarely achieves this goal. This is because such an immunogen presents the linear peptide with too much conformational freedom, such that the peptide may adopt a loose structure that either is not well recognised by the immune system, or may be entirely different to the conformation adopted by the peptide in the context of the flanking regions of the full length native protein.

In order to overcome this conformational freedom problem, it is known to design peptides in a constrained manner, by chemical interactions between two distant amino acid residues, such that the peptide is held in a curved structure which closely resembles the curve in which the peptide would be held by the flanking sequences in the full length native protein (U.S. Pat. No. 5,939,383; Hruby et al., 1990, Biochem J., 268, 249-262). To do this it is most common to incorporate two cysteine residues into the peptide sequence between which the desired intramolecular disulphide bridge forms after gentle oxidation of a dilute solution of the peptide.

The cyclised peptide thus formed is commonly conjugated to a protein carrier to form an immunogen by one of several chemistry methods. Examples of known chemistries include conjugation of amino groups between the peptide and carrier by amino reactive agents such as glutaraldehyde or formaldehyde; or condensing carboxyl groups and amino groups with carbodiimide reagents or alternatively by converting n-terminal (x-hydroxy groups to aldehydes by an oxidation reaction and conjugating this group to an amino or oxamino moiety. However, each of these chemistries has disadvantages, including a need for relatively harsh oxidative reaction conditions, poor controllability at industrial levels, formation of polymers, or not being suitable for peptides that contain specific internal amino acids (especially: Lysine, Aspartic acid, Glutamic acid, Tryptophan, Tyrosine or Serine) that could also interfere with the chemistry in an inappropriate manner.

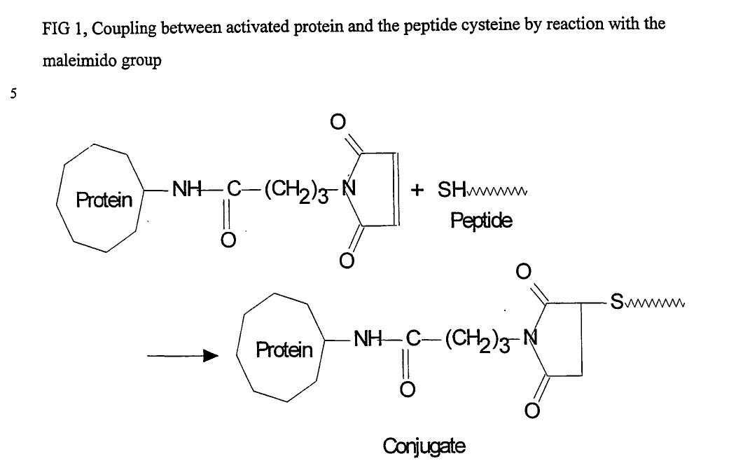

It is common, therefore, to use thio-ether linkage to conjugate peptides to protein carriers. The most common method to achieve this conjugation is to add a moiety with a terminal thiol group onto the peptide, most commonly by adding a cysteine, and then to react the reactive thiol group with a maleimide-derivatised protein carrier (Friede et al., 1994, Vaccine, 12, 791-797), for a schematic summary see FIG. 1.

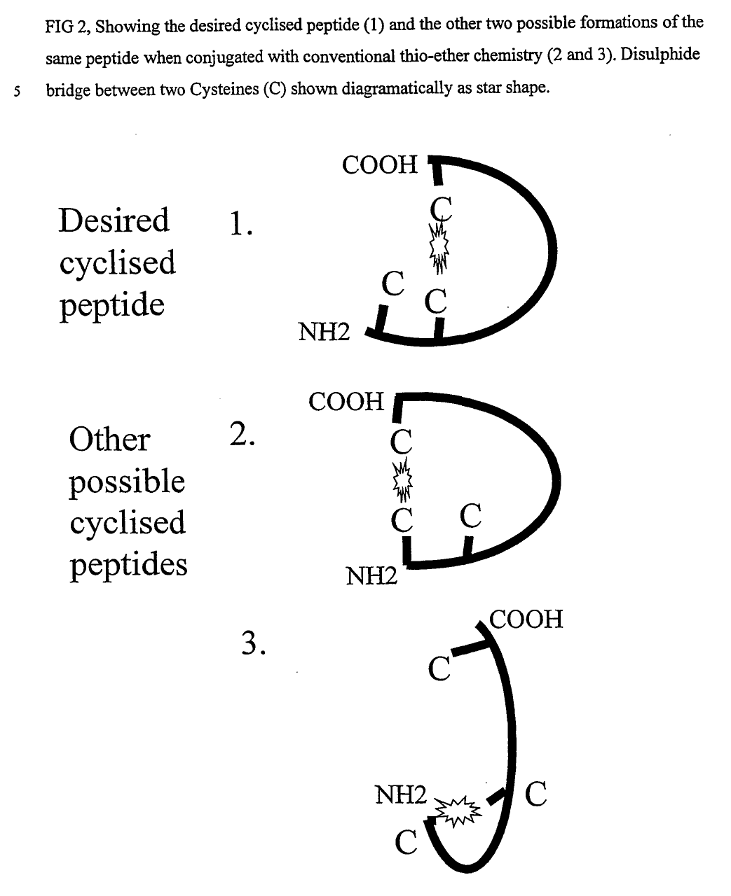

However, in the case of peptides containing an internal disulphide bond this commonly preferred peptide chemistry may have problems because of the posibility of internal disulphide rearrangement, or external rearrangement of disulphide bonds between between two adjacent peptides. In some cases the presence of a third cysteine causes unwanted interference with the disulphide bond, and a thiol-disulfide exchange can occur such that the resultant intermediate cyclised peptide product is a mixture of three possible disulphide bridge cyclised peptides (reassortant intermediates, see FIG. 2), or may additionally comprise peptide dimers or polymers.

In the case of conjugation of these peptide intermediates to a maleimide activated carrier protein, each of the reassortant intermediates is equally reactive with the reactive carrier protein, and as such they will all conjugate to the carrier. As a result, the purity of the desired product is decreased, and use of this mixture of immunogens may result in immune responses that may not, or only weakly, cross react with the epitope on the full native protein that the peptide was intended to mimic. In order to overcome these problems several authors have replaced the disulphide bond stabilised cyclic peptides, by thio-ether bonds. For example, in Ivanov et al., 1995, Bioconjugate Chemistry, 6, 269-277, one cystein is replaced by a trifunctional bromoacetyl-derivitised amino acid, thus permiting cyclisation via a non-reversible thioether bond. In such thio-ether cyclised peptides, however, the resulting peptide is fundamentally different to the original disulphide-cyclised peptide, and has a different structure which may not resemble the disulphide-cyclised peptide. Hence antibodies formed against the thio-ether cyclised peptide may not recognise the parent peptide as efficiently as antibodies formed against the disulphide-cyclised peptides.

The present invention overcomes the problems of forming a thio-ether linkage between a disulphide cyclised peptide and a carrier by providing a chemistry that does not use a terminal thiol containing group on the cyclised peptide, but instead uses another reactive group on the peptide, which may then be reacted with a thiolated carrier protein to form a thio-ether bond.

Therefore, in the present invention, there is provided a process for the manufacture of a vaccine immunogen comprising conjugating a disulphide bridge cyclised peptide to an immunogenic carrier comprising, (a) adding to a disulphide cyclised peptide a moiety comprising a reactive group which is capable of forming thio-ether linkages with thiol bearing carriers, and (b) reacting the activated cyclised peptide thus formed with a thiol bearing immunogenic carrier.

The process of the present invention overcomes the problems of internal and external disulphide rearrangement, and in addition provides conjugated products wherein the disulphide cyclised peptides are in the desired conformation. In a preferred process of the present invention, a peptide is synthesised containing two cysteine residues which are allowed to form a disulphide bridge, followed by the addition of the reactive group. The activated peptide, thus obtained, is then reacted with the thiol bearing carrier.

The reactive groups that are suitable for use in the present invention include any group which is capable forming thio-ether linkages with thiolated carriers. As which will be apparent to the man skilled in the art, preferred reactive groups may be selected from active imides, especially maleimides, haloalkyl groups such as iodoalkyl or bromoalkyl groups. Preferably the bromoalkyl group is a bromoacetyl group. The use of maleimide to link linear peptides to thiolated polymer is described in Van Dijk-Wolthius et al., 1999, Bioconjugate Chemistry, 10, 687-692. Use of bromoacetyl groups to link peptides to carriers is described in Ivanov et al., 1995, Bioconjugate Chemistry, 6, 269-277 and U.S. Pat. No. 5,444,150. Conjugation of proteins to thiolated solid phase supports for diagnostic assays is described in EP 0 396 116 A.

It is a particularly preferred aspect of the present invention when the process uses maleimide as the reactive group. Accordingly, a preferred process for conjugating a disulphide bridge cyclised peptide to a carrier comprises, (a) adding to a disulphide cyclised peptide a moiety comprising a maleimide group, and (b) reacting the activated cyclised peptide thus formed with a thiol bearing carrier. The product of this process (A conjugate suitable for use in a vaccine) forms an aspect of the present invention, and has the formula (I):

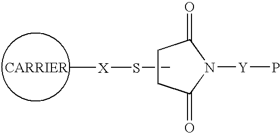

wherein, Carrier is a carrier molecule, X is either a linker or a bond, Y is either a linker or a bond, and P is a disulphide bridge cyclised peptide. When X is a bond, it should be understood that the carrier is directly linked to the sulphur atom S. Similarly, when Y is a linker it should be understood that the disulphide-bridge cyclised peptide is linked directly to the nitrogen atom N. A “linker” refers to a suitable linker group. When X is a linker group an example is the group —NHCO(CH₂)₂—. When Y is a linker group, an example is —(CH₂)₃—CONH—. It will also be clear to the man skilled in the art, that Formula (I) covers conjugates where the sulphur atom (S) is joined onto the imide ring to either of the two adjacent non-carbonyl carbon atoms, such that the conjugate may comprise the following structures:

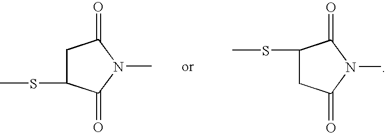

Forming an aspect of the invention is the intermediate to the process of the present invention, which is a disulphide cyclised peptide which bears a reactive group which is capable forming thio-ether linkages with thiolated carriers. Preferably said intermediate comprises a disulphide bridge cyclised peptide linked to an active imide group, in particular a maleimide group. The high purity of the final conjugated product derives from the fact that any internal or external rearrangement that occurs between the disulphide bridge and the thio-ether reactive group is irreversible, and consequently these reassortant intermediates are not reactive with the thiolated carrier protein. Only the activated peptide intermediates that have the disulphide bridge at the desired location (i.e. between the cysteines present in the peptide) with the free reactive group participate in the conjugation reaction with the thiolated carrier, thereby forming a conjugate of extremely high purity which contains cyclised peptides of the desired conformation.

Preferred maleimide derivatisation reagents are gamma-maleimidobutyric acid N-hydroxysuccinimide ester (GMBS, Molecular Formula: C₁₂H₁₂N₂O₆ Fujiwara, K., et al., J. Immunol. Meth., 45, 195-203 (1981), Tanimori, H., et al., J. Pharmacobiodyn., 4, 812-819 (1981); H. Tanimori, et al., J. Immunol. Methods 62, 123 (1983); M.D. Partis, et al., J. Prot. Chem. 2, 263 (1983); L. Moroder, et al., Biopolymers 22, 481 (1983); S. Hashida, et al., J. Appl. Biochem. 6, 56 (1984); S. Inoue, et al., Anal. Lett. 17, 229 (1984); E. Wünsch, et al., Biol. Chem. Hoppe-Seyler 366, 53 (1985)), which can be purchased from the Sigma or Pierce companies. It will be recognised that many maleimide-derivitisation reagents exist and can be used, and the addition of the maleimide group to the cyclised peptide can be performed during peptide synthesis using reagents compatible with organic synthesis, or after peptide synthesis using reagents commonly used for derivitising peptides and proteins with maleimide groups.

The process, intermediates and products of the present invention are preferably used in the manufacture of immnunogens for use in vaccines. The peptides for conjugation may be selected from any antigen against which is desired to create an immune response. The peptide may be derived from a pathogen, such as a virus, bacterium, parasite such as a worm etc.

Equally the peptide may be selected from a self protein, for example in the vaccine therapy of cancer or allergy.

In an allergic response, the symptoms commonly associated with allergy are brought about by the release of allergic mediators, such as histamine, from immune cells into the surrounding tissues and vascular structures. Histamine is normally stored in mast cells and basophils, until such time as the release is triggered by interaction with allergen specific IgE. The role of IgE in the mediation of allergic responses, such as asthma, food allergies, atopic dermatitis, type-I hypersensitivity and allergic rhinitis, is well known. On encountering an antigen, such as pollen or dust mite allergens, B-cells commence the synthesis of allergen specific IgE. The allergen specific IgE then binds to the FcεRI receptor (the high affinity IgE receptor) on basophils and mast cells. Any subsequent encounter with allergen leads to the triggering of histamine release from the mast cells or basophils, by cross-linking of neighbouring IgE/FcεRI complexes (Sutton and Gould, Nature, 1993, 366: 421-428; EP 0 477 231 B1).

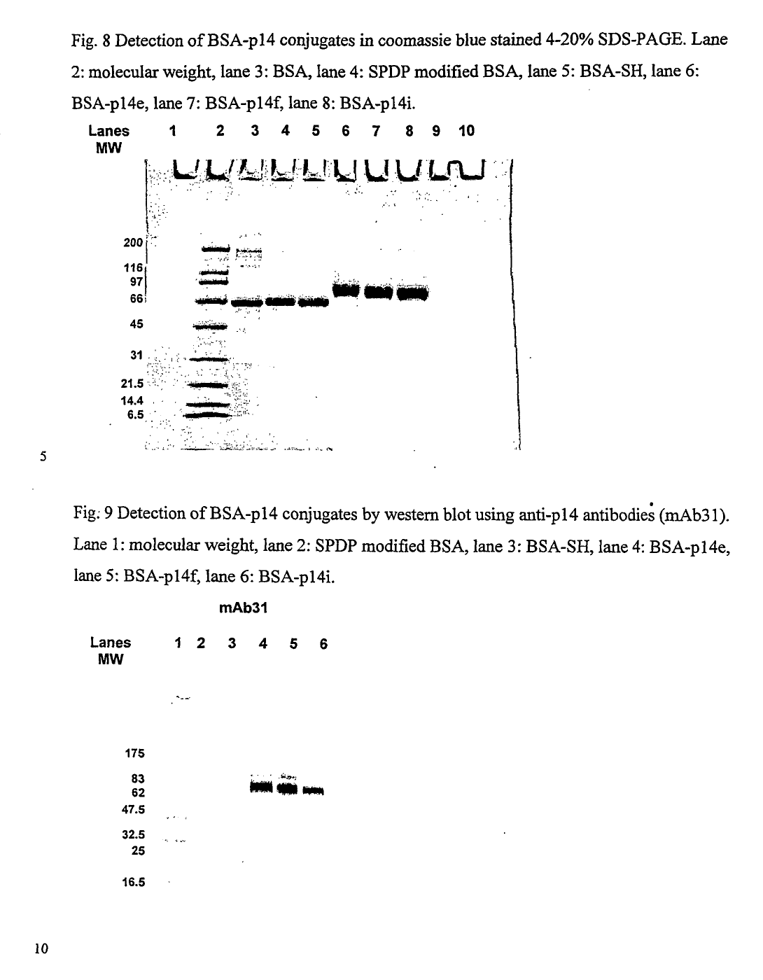

IgE, like all immunoglobulins, comprises two heavy and two light chains. The ε heavy chain consists of five domains: one variable domain (VH) and four constant domains (Cε1 to Cε4). The molecular weight of IgE is about 190,000 Da, the heavy chain being approximately 550 amino acids in length. The structure of IgE is discussed in Padlan and Davis (Mol. Immunol., 23, 1063-75, 1986) and Helm et al., (2IgE model structure deposited Feb. 2, 1990 with PDB (Protein Data Bank, Research Collabarotory for Structural Bioinformatics; http:pdb-browseres.evi.ac.uk)). Each of the IgE domains consists of a squashed barrel of seven anti-parallel strands of extended (β-) polypeptide segments, labelled a to f, grouped into two β-sheets. Four β-strands (a, b,d & e) form one sheet that is stacked against the second sheet of three strands (c,f & g) (see FIG. 8). The shape of each β-sheet is maintained by lateral packing of amino acid residue side-chains from neighbouring anti-parallel strands within each sheet (and is further stabilised by main-chain hydrogen-bonding between these strands). Loops of residues, forming non-extended (non-β-) conformations, connect the anti-parallel β-strands, either within a sheet or between the opposing sheets. The connection from strand a to strand b is labelled as the A-B loop, and so on. The A-B and d-e loops belong topologically to the four-stranded sheet, and loop f-g to the three-stranded sheet. The interface between the pair of opposing sheets provides the hydrophobic interior of the globular domain. This water-inaccessible, mainly hydrophobic core results from the close packing of residue side-chains that face each other from opposing β-sheets.

In the past, a number of passive or active immunotherapeutic approaches designed to interfere with IgE-mediated histamine release mechanism have been investigated. These approaches include interfering with IgE or allergen/IgE complexes binding to the FcεRI or FcεRII (the low affinty IgE receptor) receptors, with either passively administered antibodies, or with passive administration of IgE derived peptides to competitively bind to the receptors. In addition, some authors have described the use of specific peptides derived from IgE in active immunisation to stimulate histamine release inhibiting immune responses.

Therefore, in order to be effective, the peptide vaccines need to be able to mimic specific sites of IgE very efficiently. The preferred immunogens of the present invention, therefore, are based on peptides derived from IgE and which are capable of triggering an immune response which inhibits histamine release from basophils.

Much work has been carried out to identify specific anti-IgE antibodies which do have some beneficial effects against IgE-mediated allergic reaction (WO 90/15878, WO 89/04834, WO 93/05810). Attempts have also been made to identify epitopes recognised by these useful antibodies, to create peptide mimotopes of such epitopes and to use those as immunogens to produce anti-IgE antibodies.

WO 97/31948 describes an example of this type of work, and further describes IgE peptides from the Cε3 and Cε4 domains conjugated to carrier molecules for active vaccination purposes. These immunogens may be used in vaccination studies and are said to be capable of generating antibodies which subsequently inhibit histamine release in vivo. In this work, a monoclonal antibody (BSW17) was described which was said to be capable of binding to IgE peptides contained within the Cε3 domain which are useful for active vaccination purposes.

EP 0 477231 B 1 describes immunogens derived from the Cε4 domain of IgE (residues 497-506, also known as the Stanworth decapeptide), conjugated to Keyhole Limpet Haemocyanin (KLH) used in active vaccination immunoprophylaxis. WO 96/14333 is a continuation of the work described in EP 0 477 231 B 1.

Other approaches are based on the identification of peptides derived from Cε3 or Cε4, which themselves compete for IgE binding to the high or low affinity receptors on basophils or mast cells (WO 93/04173, WO 98/24808, EP 0 303 625 B1, EP 0 341290).

Accordingly in a preferred aspect of the present invention the process, peptide intermediates, immunogens and vaccines, comprise a peptide selected from human IgE. Preferably the disulphide bridge cyclised peptides used in the present invention are designed from the group of peptides listed in table 1. The peptides in table 1, reflect a specific area of the IgE molecule against which it is desired to generate an immune response. The peptides, therefore, constitute a starting point from which a cyclised peptide may be designed, and accordingly they either do not contain a cysteine residue, or contain a single cysteine, or contain two cysteines which may not form a disulphide bridge. Suitable peptides for use in the process or immunogens of the present invention may be designed by the addition of at least one cysteine residue to the following peptides:

TABLE 1

IgE peptides suitable to be cyclised
and used in the process of the present invention

| | |
|---|---|
| Peptide sequence | SEQ ID NO. |

| | |
|---|---|
| EDGQVMDVD | 1 |
| STTQEGEL | 2 |
| SQKHWLSDRT | 3 |
| GHTFEDSTKK | 4 |
| GGGHFPPT | 5 |
| PGTINI | 6 |
| FTPPT | 7 |
| CLEDGQVMDVDLL | 8 |
| LLDVDMVQGDELC | 9 |
| WLEDGQVMDVDLC | 10 |
| QVMDVDL | 11 |
| LEDGQVMDVD | 12 |
| CSTTQEGELA | 13 |
| TTQEGE | 14 |
| CSQKHWLSDRT | 15 |
| TYQGHTFEDSTKKCADSNPRGV | 16 |
| GGHFPP | 17 |
| CCVADPETQMTPSSEMF | 18 |
| CCVADPETQMTPSSEMF | 19 |
| CCVTDVQTTNMDVPAGQ | 20 |
| TCCVTDIPPPDYEQSLG | 21 |
| CCESDIPLNELHALADP | 22 |
| CCKSDIPSPVTQFNTMK | 23 |
| CCQSDVPHQPGINDLHV | 24 |
| CCMSDTPDISRLPVPDS | 25 |
| CCMSDSPADPNRGLPIW | 26 |
| CCLSDDAPTLPVRR | 27 |
| CCITDVPQGVMYKGSPD | 28 |
| ECKVDGQLSDSPLLRNN | 29 |
| CCMTDDPMDPNSTWAIR | 30 |
| CCMTDDPMYTNSTWAIR | 31 |
| CCVDDTPNSGLAMRVSK | 32 |
| CCEVDDFPTHHPGWTLR | 33 |
| SCNLNHQSCDIPPVKQI | 34 |
| CCMADQELDLGHNAANA | 35 |
| CCVMDLELASGF | 36 |
| CCVMDIEVRGSA | 37 |
| CCQRDVELVFGS | 38 |
| CCRADFEVGNGG | 39 |
| CCVSDEPAGVRD | 40 |
| GAGWQEKDKELR | 41 |
| GAMTAGQLSDLP | 42 |
| VAGGQVVDRELK | 43 |
| KAGEQAMDMELR | 44 |
| RGRNQIMDLEI | 45 |
| QIDRQITDTLL | 46 |
| REQQISDVPRV | 47 |
| CQAMDAEILNQV | 48 |
| GQMMDTELLNR | 49 |
| SMEGQVRDIQV | 50 |
| YQQRDLELLAE | 51 |
| SMGQKVDRELV | 52 |
| SMGQEVDRELV | 53 |
| AENDQMVDWEI | 54 |
| GGWQESDIPGR | 55 |
| GGWQEKDKELR | 56 |
| HCCRIDREVSGA | 57 |
| DCDWINPPDPPHFWKDT | 58 |
| DALDERAWRARA | 59 |
| RASGKPVNHSTRKEEKQRNGTL | 60 |
| GTRDWIEGE | 61 |
| PHLPRALMRSTTKTSGPRA | 62 |
| PEWPGSRDKRT | 63 |
| EQKDE | 64 |
| LSRPSPFDLFIRKSPTITC | 65 |
| WLHNEVQLPDARHSTTQPRKT | 66 |
| CRASGKPVNHSTRKEEKQRNGLL | 67 |
| GKPVNHSTGGC | 68 |
| GKPVNHSTRKEEKQRNGC | 69 |
| CGKPVNHSTRKEEKQRNGLL | 70 |
| RASGKPVNHSTGGC | 71 |
| CGTRDWIEGLL | 72 |
| CGTRDWIEGETL | 73 |
| GTRDWIEGETGC | 74 |
| CHPHLPRALMLL | 75 |
| CGTHPHLPRALM | 76 |
| THPHLPRALMRSC | 77 |
| GPHLPRALMRSSSC | 78 |
| APEWPGSRDKRTC | 79 |
| APEWPGSRDKRTLAGGC | 80 |
| CGGATPEWPGSRDKRTL | 81 |
| CTRKDRSGPWEPA | 82 |
| CGAEWEQKDEL | 83 |
| AEWEQKDEFIC | 84 |
| GEQDKEFIC | 85 |
| CAEGEQKDEL | 86 |
| LFIRKS | 87 |
| PSKGTVN | 88 |
| LHNEVQLPDARHSTTQPRKTKGS | 89 |
| SVNPGK | 90 |
| CPEWPGCRDKRTG | 91 |
| TPEWPGCRDKRCG | 92 |
| DPEWPGSRDKKGSC | 93 |
| DWPGSRDKRKGSC | 94 |
| DATPEWPGSRDKRTLKGSC | 95 |

Accordingly examples of peptides listed in table 1, which have been modified to be specific disulphide bridge cyclised peptides suitable for the present invention are listed in table 2.

TABLE 2

modified cyclic peptides.

| | |
|---|---|
| Peptide sequence | SEQ ID NO. |

| | |
|---|---|
| CLEDGQVMDVDLC | 96 |
| CFINKQMADLELCPRE | 97 |
| CFMNKQLADLELCPRE | 98 |
| CLEDGQVMDVDLCPREAAEGDK | 99 |
| CLEDGQVMDVDLCGGSSGGP | 100 |
| CLEDGQVMDVDCPREAAEGDK | 101 |
| KCREVWLGESETIMDCE | 102 |
| ACREVWLGESETIMDCD | 103 |
| SCREVWLGESETVMDCG | 104 |
| NCQDLMLREDAGCWSKM | 105 |
| DCEEPMCSPVLLQQLKL | 106 |
| CFINKQMADLELC | 107 |
| CFMNKQLADLELC | 108 |
| KCREVWLGESETIMDC | 109 |
| HCQQVFFPQDYLWCQRG | 110 |
| SCREVWLGGSEMIMDCE | 111 |
| ECNQNLSGSLRHVDLNC | 112 |
| DCEEPMCSPVLLQKLKP | 113 |
| SCREVWLGGSEMIMDCE | 114 |
| RCDQQLPRDSYTFCMMS | 115 |
| SCPAFPREGDLCAPPTV | 116 |
| FCPEPICSPPLSRMTLS | 117 |
| VCDECVSRELAL | 118 |
| WCLEPECAPGLL | 119 |
| VCDECVSRELAL | 120 |
| DCLSKGQMADLC | 121 |
| SCQGREVRRECW | 122 |
| WCREVWLGESETIMDCE | 123 |
| ACREVWLGESETIMDCD | 124 |
| GCAEPKCWQALHQKLKP | 125 |
| ECRGPNMQMQDHCPTTD | 126 |
| QCNAVLEGLQMVDHCWN | 127 |
| HCKNEFKKGQWTYSCSD | 128 |
| QCRQFVMNQSEKEFGQC | 129 |
| NCFMNKQLADLELCPRE | 130 |
| SCAYTAQRQCSDVPNPG | 131 |
| GCFMNKQMADLELCPRTAA | 132 |
| ACFMNKQMADLELCPRVAA | 133 |
| GCFINKQLADLELCPRVAA | 134 |
| GCFMNKQLADWELCPRAAA | 135 |
| ECFMNKQLADSELCPRVAA | 136 |
| GCFMNKQLADPELCPREAE | 137 |
| GCFMNKQLVDLELCPRGAA | 138 |
| GCFMNKQLADLELCPREAA | 139 |
| GCFMNKQQADLELCPRGAA | 140 |
| GCFINKQMADLELCPREAA | 141 |
| CLEDGQVMDVDCPREAAEGD | 142 |
| CLEDGQVMDVDLCPREAAEGD | 143 |
| QCNAVLEGLQMVDHCWN | 144 |
| ECLKIEQQCADIVEIPR | 145 |
| SCAYTAQRQCSDVPNPG | 146 |
| ECRGPNMQMQDHCPTTD | 147 |
| ECLVYGQMADCAAGGWP | 148 |
| QCRQFVMNQSEKEFGQC | 149 |
| HCKNEFKKGQWTYSCSD | 150 |
| CAPGMGCWESVK | 151 |
| SCREVWLGGSEMIMDCE | 152 |
| SCPAFPREGDLCAPPTV | 153 |
| FCPEPICSPPLSRMTLS | 154 |
| ECNQNLSGSLRHVDLNC | 155 |
| RCDQQLPRDSYTFCMMS | 156 |
| HCQQVFFPQDYLWCQRG | 157 |
| DCEEPMCSPVLLQKLKP | 158 |
| NCQDQMLREDAGCWSKI | 159 |
| HCEEPEYSPATRVFCGR | 160 |
| ACFSRNGQVTDVPHSCY | 161 |
| KCPTYPKPNDRCLWPVP | 162 |
| YCPKYPLEGDCLLDNDY | 163 |
| RCEEWLCIPPAPAFAPP | 164 |
| TCGQSELCASLETHHV | 165 |
| NCNDNPMLDCMPAWSS | 166 |
| SCQGREVRRECW | 167 |
| VCDECVSRELAL | 168 |
| WCLEPECAPGLL | 169 |
| DCLSKGQMADLC | 170 |
| VCDECVSRELAL | 171 |
| GCPTWPRVGDHC | 172 |
| RCQSARVVPECW | 173 |
| SCAPSGDCGYKG | 174 |
| GCPMWPQPDDEC | 175 |
| ECPRWPLMGDGC | 176 |
| GCQVGELVWCRE | 177 |
| QCVRDGTRKVCM | 178 |
| TCLVDRQESDVC | 179 |
| DCVVDGDRLVCL | 180 |
| RCEQGALRCVGE | 181 |
| VCPPGWKNLGCN | 182 |
| MCQGWEIVSECW | 183 |
| ADGAGCFMNKQMADLELCPREAAEA | 184 |
| ADGAGCFMNKQMADLELCPRTAAEA | 185 |
| ADGAGCFMNKQMADLELCPRVAAEA | 186 |
| ADGAGCFINKQLADLELCPRVAAEA | 187 |
| ADGAGCFINKQMADLELCPREAAEA | 188 |
| ADGAGCFMNKQMADLEMCPRDDAEA | 189 |
| ADGAGCFMNKQLADPELCPREAEEA | 190 |
| ADGAGCFMNKQLVDLELCPRGAAEA | 191 |
| ADGAGCFMNNQLADWELCPRAAAEA | 192 |
| ADGAGCFMNKQMADWEMCPRAAAEA | 193 |
| ADGAGCFMNKQQADLELCPRGAAEA | 194 |
| ADGAECFMNKQLADSELCPRVAAEA | 195 |
| ADGAGCFMNKQLADLELCPREAAEA | 196 |
| ADGAGCFINMQMADQELCPRAAAEA | 197 |
| ADGAGCFINKQMSDFELCPREAGEA | 198 |
| ADGAGCFINKQMADLELCTREAAEA | 199 |
| ADGAGCFINKQMADLELCPRQAAEA | 200 |
| ADGAGCFINNQMADLELCPRGGAEA | 201 |
| ADGAGCFINKQMADWELCPREGAEA | 202 |
| ADGAGCFINKQMADLELCPSQAAEA | 203 |
| ADGAGCFINKQMADLELCPREGAEA | 204 |
| ADGAGCFINKQMADSELCPREPAEA | 205 |
| ADGAGCFIKKQMADLELCPREAWEA | 206 |
| ADGAECFINKQMADRELCAREVAEA | 207 |
| ADGAGCFIDKQMADLELCPRAAAEA | 208 |
| ADGAGCFINKQMADLELCRREAGEA | 209 |
| ADGAGCFKNKQMVDSELCARQAAEA | 210 |
| ADGAGCFQNKQMADLELCPREAAEA | 211 |
| ADGAECFINKQRADLELCPGEAAEA | 212 |
| ADGAGCFINKQMADSELCPAAAAEA | 213 |
| ADGAGCFINRQMADPELCPREAAEA | 214 |
| ADGAGCFIEKQMADMELCQARAAEA | 215 |
| ADGAGCFINKQMADWELCPREAAEA | 216 |
| ADGAGCFINNQMADLELCPREAAEA | 217 |
| ADGAGCFIEKQMADMELCQRETAEA | 218 |
| ADGAGCFINKQMADMELCPREAAEA | 219 |
| ADGAGCFINKQMADLELCPREAAEA | 220 |
| ADGAGCFRNKQMADLELCPREAAEA | 221 |
| ADGAGCFRNKQMADLELCPREAAEA | 222 |
| ADGAGCFINRQLADMELCSRGAAEA | 223 |
| ADGAECFINRQMADLELCGREAAEA | 224 |
| ADGAGCFISPQLADWKRCMREAAEA | 225 |
| ADGAGCSIHTQMADWERCLREGAEA | 226 |
| ADGAGCSIHRQMADWERCLREGAEA | 227 |
| CSSCDGGGHKPPTIQC | 228 |
| CLQSSCDGGGHFPPTIQLLC | 229 |
| APCWPGSRDCRTLAG | 230 |
| ACPEWPGSRDRCTLAG | 231 |
| CATPEWPGSRDKRTLCG | 232 |
| CATPEWPGSRDKRTCG | 233 |
| TPCWPGSRDKRCG | 234 |
| CSRPSPFDLFIRKSPTITC | 235 |
| CSRPSPFDLFIRKSPTIC | 236 |
| CSRPSPFDLFIRKSPTC | 237 |
| CSRPSPFDLFIRKSPC | 238 |
| CRPSPFDLFIRKSPC | 239 |
| CRPSPFDLFIRKSPTC | 240 |
| CRPSPFDLFIRKSPTIC | 241 |
| CRPSPFDLFIRKSPTITC | 242 |
| CPSPFDLFIRKSPTITC | 243 |
| CPSPFDLFIRKSPTIC | 244 |
| CPSPFDLFIRKSPTC | 245 |
| CPSPFDLFIRKSPTC | 246 |
| CYAFATPEWPGSRDKRTLAC | 247 |
| CYAFATPEWPGSRDKRTLC | 248 |
| CYAFATPEWPGSRDKRTC | 249 |
| CYAFATPEWPGSRDKRC | 250 |
| CAFATPEWPGSRDKRC | 251 |
| CAFATPEWPGSRDKRTC | 252 |
| CAFATPEWPGSRDKRTLC | 253 |
| CAFATPEWPGSRDKRTLAC | 254 |
| CFATPEWPGSRDKRTLAC | 255 |
| CFATPEWPGSRDKRTLC | 256 |
| CFATPEWPGSRDKRTC | 257 |
| CFATPEWPGSRDKRC | 258 |
| CTWSRASGKPVNHSTRC | 259 |
| CTWSRASGKPVNHSTC | 260 |
| CTWSRASGKPVNHSC | 261 |
| CTWSRASGKPVNHC | 262 |
| CWSRASGKPVNHC | 263 |
| CWSRASGKPVNHSC | 264 |
| CWSRASGKPVNHSTC | 265 |
| CWSRASGKPVNHSTRC | 266 |
| CSRASGKPVNHSTRC | 267 |
| CSRASGKPVNHSTC | 268 |
| CSRASGKPVNHSC | 269 |
| CSRASGKPVNHC | 270 |
| CQWLHNEVQLPDARHSC | 271 |
| CQWLHNEVQLPDARHC | 272 |
| CQWLHNEVQLPDARC | 273 |
| CQWLHNEVQLPDAC | 274 |
| CWLHNEVQLPDAC | 275 |
| CWLHNEVQLPDARC | 276 |
| CWLHNEVQLPDARHC | 277 |
| CWLHNEVQLPDARHSC | 278 |
| CLHNEVQLPDARHSC | 279 |
| CLHNEVQLPDARHC | 280 |
| CLHNEVQLPDARC | 281 |
| CLHNEVQLPDAC | 282 |
| CPSPFDLFIRKSPCGSK | 283 |
| CPSPFDLFIRKSPTCGSK | 284 |
| FAGCSRASGKPVNHCGAAEG | 285 |
| FAGCSRASGKPVNHSCGAAEG | 286 |
| FAGCSRASGKPVNHSTCGAAEG | 287 |
| FAGCSRASGKPVNHSTRCGAAEG | 288 |
| CSRASGKPVNHCGSK | 289 |
| CSRASGKPVNHSCGSK | 290 |
| CSRASGKPVNHSTCGSK | 291 |
| FAGCFATPEWPGSRDKRCGAAEG | 292 |
| FAGCFATPEWPGSRDKRTCGAAEG | 293 |
| FAGCFATPEWPGSRDKRTLCGAAEG | 294 |
| FAGCFATPEWPGSRDKRTLACGAAEG | 295 |
| CPEWPGSRDKRCGSK | 296 |
| CWPGSRDKRCGSK | 297 |
| CPEWPGSRDKRCGAAEG | 298 |
| FAGCLHNEVQLPDACGAAEG | 299 |
| FAGCLHNEVQLPDARCGAAEG | 300 |
| FAGCLHNEVQLPDARHCGAAEG | 301 |
| FAGCLHNEVQLPDARHSCGAAEG | 302 |
| FAGCLHNEVQLPDASGAAEG | 303 |
| CPEWPGSRDRCGSK | 304 |
| CWPGSRDRRCGSK | 305 |
| CDSNPRGVSAADSNPRGVSC | 306 |
| CLVVDLAPSKGTVNC | 307 |
| CKQRNGTLC | 308 |
| CEEKQRNGTLTVC | 309 |
| CHPHLPRC | 310 |
| CTHPHLPRAC | 311 |
| CVTHPHLPRALC | 312 |
| CRVTHPHLPRALMC | 313 |
| CXRVTHPHLPRALMRC | 314 |
| CQXRVTHPHLPRALMRSC | 315 |
| CYQXRVTHPHLPRALMRSTC | 316 |
| CPEWPGSRDKRC | 317 |
| CRQRNGTLC | 318 |
| CEERQRNGTLTVC | 319 |
| CMRVTHPHLPRALMRC | 320 |
| CQMRVTHPHLPRALMRSC | 321 |
| CYQMRVTHPHLPRALMRSTC | 322 |
| ACPEWPGSRDRCTLAG | 323 |
| GGCLEDGQVMDVDC | 324 |
| CLEDGQVMDCGSK | 325 |
| CLEDGQVMDVDLCGSK | 326 |
| CLEDGQVMDVDLCPREAAEGDK | 327 |
| CLEDGQVMDVDLCGGSSGGK | 328 |

Immunogens produced by the process of the present invention which may incorporate the modified peptides of table 1, or the cyclic peptides of table 2, form a preferred aspect of the present invention. Mimotopes which have the same characteristics as these peptides, and immunogens comprising such mimotopes which generate an immune response which cross-react with the IgE epitope in the context of the IgE molecule, also form part of the present invention. The meaning of mimotope is defined as an entity which is sufficiently similar to the native IgE peptides listed in tables 1 or 2, so as to be capable of being recognised by antibodies which recognise the native IgE peptide; (Gheysen, H. M., et al., 1986, Synthetic peptides as antigens. Wiley, Chichester, Ciba foundation symposium 119, p130-149; Gheysen, H. M., 1986, Molecular Immunology, 23,7, 709-715); or are capable of raising antibodies, when coupled to a suitable carrier, which antibodies cross-react with the native IgE epitope.

The preferred peptides to be used in the process or inmmunogens of the present invention mimic the surface exposed regions of the IgE structure, however, within those regions the dominant aspect is thought by the present inventors to be those regions within the surface exposed area which correlate to a loop structure. The structure of the domains of IgE are described in “Introduction to protein Structure” (page 304, 2^{nd} Edition, Branden and Tooze, Garland Publishing, New York, ISBN 0 8153 2305-0) and take the form a β-barrel made up of two opposing anti-parallel β-sheets (see FIG. 8). The immunogens may comprise a disulphide bridge cyclised peptide which is a sequence derived from a loop of the IgE domains. Preferred examples of this are the A-B loop of Cε3, the A-B loop of Cε4, the C-D loop of Cε3, the C-D loop of Cε4, the A-B loop of Cε2 and the C-D loop of Cε2.

Peptide mimotopes of the above-identified IgE epitopes may be designed for a particular purpose by addition, deletion or substitution of elected amino acids. Thus, the peptides of the present invention may be modified for the purposes of ease of conjugation to a protein carrier. For example, it may be desirable for some chemical conjugation methods to include a terminal cysteine to the IgE epitope. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the IgE peptide as found in the context of the whole IgE molecule. For example, the peptides may be altered to have an N-terminal cysteine and a C-terminal hydrophobic amidated tail. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids. In addition, these may be cyclised by techniques known in the art to constrain the peptide into a conformation that closely resembles its shape when the peptide sequence is in the context of the whole IgE molecule. A preferred method of cyclising a peptide comprises the addition of a pair of cysteine residues to allow the formation of a disulphide bridge.

Further, those skilled in the art will realise that mimotopes or immunogens of the present invention may be larger than the above-identified epitopes, and as such may comprise the sequences disclosed herein. Accordingly, the mimotopes of the present invention may consist of addition of N and/or C terminal extensions of a number of other natural residues at one or both ends. The peptide mimotopes may also be retro sequences of the natural IgE sequences, in that the sequence orientation is reversed; or alternatively the sequences may be entirely or at least in part comprised of D-stereo isomer amino acids (inverso sequences).

Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system (for example P14c).

Alternatively, peptide mimotopes may be identified using antibodies which are capable themselves of binding to the IgE epitopes of the present invention using techniques such as phage display technology (EP 0 552 267 B1). This technique, generates a large number of peptide sequences which mimic the structure of the native peptides and are, therefore, capable of binding to anti-native peptide antibodies, but may not necessarily themselves share significant sequence homology to the native IgE peptide. This approach may have significant advantages by allowing the possibility of identifying a peptide with enhanced immunogenic properties (such as higher affinity binding characteristics to the IgE receptors or anti-IgE antibodies, or being capable of inducing polyclonal immune response which binds to IgE with higher affinity), or may overcome any potential self-antigen tolerance problems which may be associated with the use of the native peptide sequence. Additionally this technique allows the identification of a recognition pattern for each native-peptide in terms of its shared chemical properties amongst recognised mimotope sequences.

Alternatively, peptide mimotopes may be generated with the objective of increasing the immunogenicity of the peptide by increasing its affinity to the anti-IgE peptide polyclonal antibody, the effect of which may be measured by techniques known in the art such as (Biocore experiments). In order to achieve this the peptide sequence may be electively changed following the general rules:

To maintain the structural constraints, prolines and glycines should not be replaced

Other positions can be substituted by an amino acid that has similar physicochemical properties.

As such, each amino acid residue can be replaced by the amino acid that most closely resembles that amino acid. For example, A may be substituted by V, L or I, as described in the following table 3.

| | Exemplary | Preferred |
|---|---|---|
| Original residue | substitutions | substitution |
| A | V, L, I | V |
| R | K, Q, N | K |
| N | Q, H, K, R | Q |
| D | E | E |
| C | S | S |
| Q | N | N |
| E | D | D |
| G | A | A |
| H | N, Q, K, R | N |
| I | L, V, M, A, F | L |
| L | I, V, M, A, F | I |
| K | R, Q, N | R |
| M | L, F, I | L |
| F | L, V, I, A, Y, W | W |
| P | A | A |
| S | T | T |
| T | S | S |
| W | Y, F | Y |
| Y | W, F, T, S | F |
| V | I, L, M, F, A | L |

The present invention, therefore, provides a process for the manufacture of a vaccine and novel immunogens comprising disulphide bridge cyclised peptides conjugated by the process of the present invention, and the use of the immunogens in the manufacture of pharmaceutical compositions for the prophylaxis or therapy of disease. Preferably the process and the immunogens of the present invention are used in vaccines for the immunoprophylaxis or therapy of allergies.

It is envisaged that the peptides used in the process of present invention will be of a small size. Peptides, therefore, should be less than 100 amino acids in length, preferably shorter than 75 amino acids, more preferably less than 50 amino acids, and most preferable within the range of 4 to 25 amino acids long.

The most preferred peptides for use in the processes and conjugates of the present invention are SEQ ID NO.s 99, 304, 305, 306, 307, 308, 309, 310, 311, 312, 313, 314, 315, 316, 317, 318, 319, 320, 321, 322, 323, 324, 325, 326, 327, and 328.

The types of immunogenic carriers used in the immunogens of the present invention will be readily known to the man skilled in the art. The preferred function of the carrier is to provide cytokine help in order to help induce an immune response against the IgE peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD). Alternatively, the process may be used to conjugate the cyclic peptides directly to liposome carriers, which may additionally comprise carriers capable of providing T-cell help. Preferably the ratio of peptides to carrier is in the order of 1:1 to 20:1, and preferably each carrier should carry between 3-15 peptides.

In an embodiment of the invention a preferred carrier is Protein D from Haemophilus influenzae (EP 0 594 610 B1). Protein D is an IgD-binding protein from Haemophilus influenzae and has been patented by Forsgren (WO 91/18926, granted EP 0 594610 B1). In some circumstances, for example in recombinant immunogen expression systems it may be desirable to use fragments of protein D, for example Protein D 1/3^{rd} (comprising the N-terminal 100-110 amino acids of protein D (GB 9717953.5)).

Peptides can be readily prepared using the ‘Fmoc’ procedure, utilising either polyamide or polyethyleneglycol-polystyrene (PEG-PS) supports in a fully automated apparatus, through techniques well known in the art (techniques and procedures for solid phase synthesis are described in ‘Solid Phase Peptide Synthesis: A Practical Approach’ by E. Atherton and R. C. Sheppard, published by IRL at Oxford University Press (1989)) followed by acid mediated cleavage to leave the linear, deprotected, modified peptide. This peptide can be readily oxidised and purified to yield the disulphide-bridge modified peptide, using methodology outlined in ‘Methods in Molecular Biology, Vol. 35: Peptide Synthesis Protocols (ed. M. W. Pennington and B. M. Dunn), chapter 7, pp9l-171 by D. Andreau et al.

Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, N.Y. (1989).

The amount of protein in each vaccine dose is selected as an amount which induces an immunoprotective response without significant adverse side effects in typical vaccines. Such amount will vary depending upon which specific immunogen is employed and how it is presented. Generally, it is expected that each dose will comprise 1-1000 μg of protein, preferably 1-500 μg, more preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

Vaccines of the present invention, may advantageously also include an adjuvant. Suitable adjuvants for vaccines of the present invention comprise those adjuvants that are capable of enhancing the antibody responses against the immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (for example hydroxide or phosphate salts). Preferred adjuvants for use with immunogens of the present invention include: aluminium or calcium salts (hydroxide or phosphate), oil in water emulsions (WO 95/17210, EP 0 399 843), or particulate carriers such as liposomes (WO 96/33739). Immunologically active saponin fractions (e.g. Quil A) having adjuvant activity derived from the bark of the South American tree Quillaja Saponaria Molina are particularly preferred. Derivatives of Quil A, for example QS21 (an HPLC purified fraction derivative of Quil A), and the method of its production is disclosed in U.S. Pat. No.5,057,540. Amongst QS21 (known as QA21) other fractions such as QA17 are also disclosed. 3 De-O-acylated monophosphoryl lipid A is a well known adjuvant manufactured by Ribi Immunochem, Montana. It can be prepared by the methods taught in GB 2122204B. A preferred form of 3 De-O-acylated monophosphoryl lipid A is in the form of an emulsion having a small particle size less than 0.2cm in diameter (EP 0 689 454 B1).

Adjuvants also include, but are not limited to, muramyl dipeptide and saponins such as Quil A, bacterial lipopolysaccharides such as 3D-MPL (3-O-deacylated monophosphoryl lipid A), or TDM. As a further exemplary alternative, the protein can be encapsulated within microparticles such as liposomes, or in non-particulate suspensions of polyoxyethylene ether (UK Patent Application No. 9807805.8). Particularly preferred adjuvants are combinations of 3D-MPL and QS21 (EP 0 671 948 B1), oil in water emulsions comprising 3D-MPL and QS21 (WO 95/17210, PCT/EP98/05714), 3D-MPL formulated with other carriers (EP 0 689 454 B1), or QS21 formulated in cholesterol containing liposomes (WO 96/33739), or immunostimulatory oligonucleotides (WO 96/02555). Alternative adjuvants include those described in WO 99/52549.

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule. Such booster constructs may comprise an alternative carrier or may be in the absence of any carrier.

In a further aspect of the present invention there is provided an immunogen or vaccine as herein described for use in medicine.

Preferably, the vaccine preparation of the present invention may be used to protect or treat a mammal susceptible to, or suffering from allergies, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. A preferred route of administration is via the transdermal route, for example by skin patches. Accordingly, there is provided a method for the treatment of allergy, comprising the administration of a peptide, immunogen, or ligand of the present invention to a patient who is suffering from or is susceptible to allergy.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4,474,757.

The present invention is illustrated by but not limited to the following examples.

### EXAMPLE 1

Conjugation of Disulphide Cyclisedpeptide to a Carries; by Conjugating a Maleimide Activated Peptide to Thiolated Protein D or BSA as a Carrier.

In the present example, a maleimide derivatised cyclic peptide is reacted with a thiol bearing carrier. The thiol group being generated on either Protein D (PD) or BSA as the carrier by reduction of the SPDP derivative of the carrier.

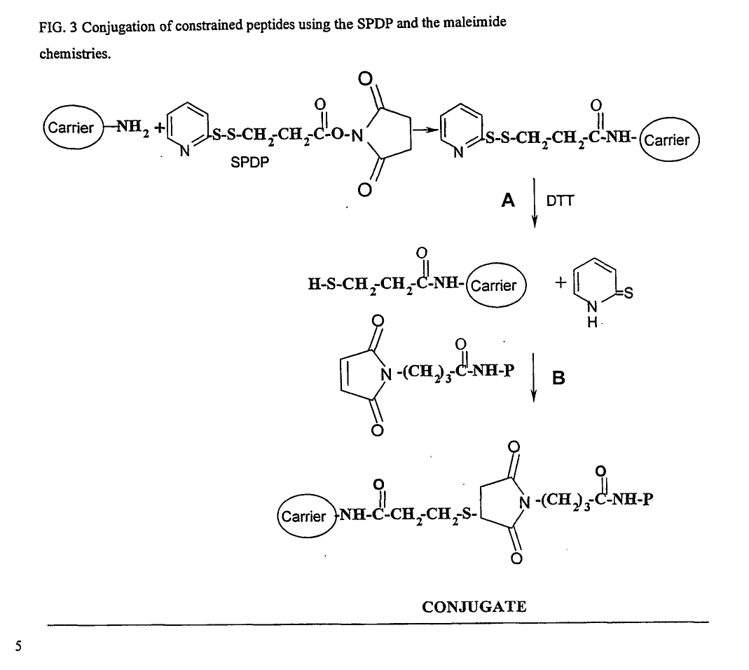

N-Succinimidyl 3-(2-pyridyldithio)propionate (SPDP) is a heterobifunctional cross-linking agent which under mild conditions, reacts by its NHS-ester group with amino groups of the protein (FIG. 3) (Hermanson G. T. Bioconjugate Techniques, 1996). NHS-ester crosslinking reactions are most commonly performed in phosphate, bicarbonate/carbonate and borate buffers. Other buffers can be used provided they do not contain primary amines. Treatment of a SPDP modified protein with DTT (Dithiothreitol, or another disulfide-reducing agent) releases the pyridine-2-thione leaving group and forms a free sulfhydryl (FIG. 3A). The reaction is generally performed with 25 mM DTT at pH 4.5 to avoid the reduction of the protein's S—S bonds. For protein not containing S—S bonds, the DTT reduction may be performed at pH 7-9. The reaction between a maleimide group added on the peptide and the sulfhydryl groups present on the carrier produces the immunogen of the present invention (FIG. 3B). The maleimide-activated peptide was obtained by reaction between the peptide (P) and a heterobifunctionnal cross-linking reagent like GMBS (gamma-maleimidobutyric acid N-hydroxysuccinimide ester).

Methods

SPDP Modified Protein

BSA (Pierce) is dissolved at a concentration of 10 mg/ml in 50 mM sodium phosphate, 0.15 M NaCl, pH 7.2. SPDP was dissolved at a concentration of 6.2 mg/ml in DMSO (makes a 20 mM stock solution). A sufficient quantity of the stock solution of SPDP was then added to the protein to be modified (for BSA, a 15 fold molar excess of SPDP over protein, and for PD, a 25 fold molar excess). After one hour at room temperature, the modified protein was purified from reaction by products by dialysis against 50 mM sodium phosphate, 10 mM EDTA pH 6.8 or by gel filtration. The sample is applied on a desalting column (Sephadex G25) equilibrated with phosphate buffer pH 6.8 (or 100 mM sodium acetate, 0.15 M NaCl, 1 mM EDTA pH 4.5 if S-S containing proteins are to be reduced in the next step). Fractions of 1 ml are collected and monitored by adsorbance at 280 nm. Fractions containing SPDP modified protein are pooled.

The number of thiopyridyl groups introduced in BSA is estimated spectrophotometrically: transfer 200 μl of modified BSA in a spectrophotometer cuvette and add 200 μl of 50 mM mercaptoethanol in 100 mM phosphate buffer, pH 7. Measure absorbance at 343 nm before and after addition of mercaptoethanol. Evaluate the quantity of thiopyridone liberated using A_{343 nm}=8000 M⁻¹cm⁻¹.

Use of DTT to Cleave Disulfide-Containing Cross-Linking Agents

DTT was added to a final concentration of 1-10 mM. Incubate for 2 h at room temperature. For removal of excess of DTT, gel filtration using Sephadex G-25 was used. To maintain the stability of the exposed sulfhydryl groups, 10 mM EDTA was included in the chromatography buffer (100 mM sodium phosphate pH 6.8). The presence of oxidized DTT can be monitored during elution by measuring the absorbance at 280 nm.

Maleimide Modified Peptide

Peptide was dissolved in 100 mM sodium phosphate pH 6.8. GMBS (Pierce) was then added to the peptide sample. A 2.5-fold molar excess of the cross-linker over the peptide was used. After 1 hr at room temperature, reaction by-products were removed by gel filtration using a sephadex G-10 (100 mM sodium phosphate pH 6.8). Fractions of 1 ml were collected and monitored by adsorbance at 280 nm. Presence of maleimide group was demonstrated by Ellman's reaction.

Reaction Between SPDP Modified Protein and Maleimide Activated Peptide

An excess of maleimide activated peptide (about 22 fold molar excess of maleimide activated peptide over the protein) was added to the SPDP modified protein and was agitated during 1 hr at room temperature followed by three dialysis against 100 mM Na phosphate pH 6.8. After filtration through 0.2 μm pore size (millipore filter), protein content was estimated by Lowry.

Results

1. Obtention of the SPDP Modified Protein

Several assays were conducted with different concentrations of SPDP using BSA or PD as carrier.

1 .a Assays on PD

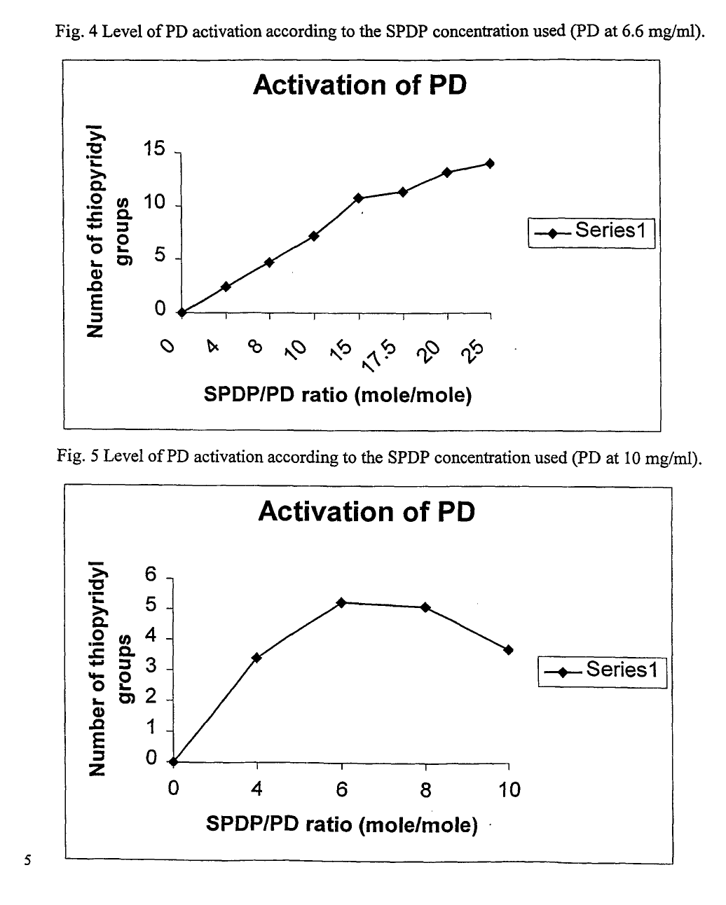

The number of thiopyridyl groups introduced was estimated spectrophotometrically by evaluation of thiopyridone liberated after addition of mercaptoethanol. Several assays were realized using PD at a concentration of 6.6 mg/ml or 10 mg/ml. results At least 14 thiopyridyl groups could be introduced on PD (FIG. 4). However, at a concentration of 10 mg/ml of PD only 4-5 thiopyridyl groups could be introduced on PD (FIG. 5). Indeed, precipitation of PD was observed when assays to obtain more thiopyridyl groups were carried out. However, this precipitation is partially induced by DMSO used to dissolve SPDP (6.2 mg/ml). This problem could be resolved by using the water-soluble sulfo-LC-SPDP (Sulfosuccinimidyl 6-[2-pyridyldithio)-propionamido]hexanoate).

1.b Assays on BSA

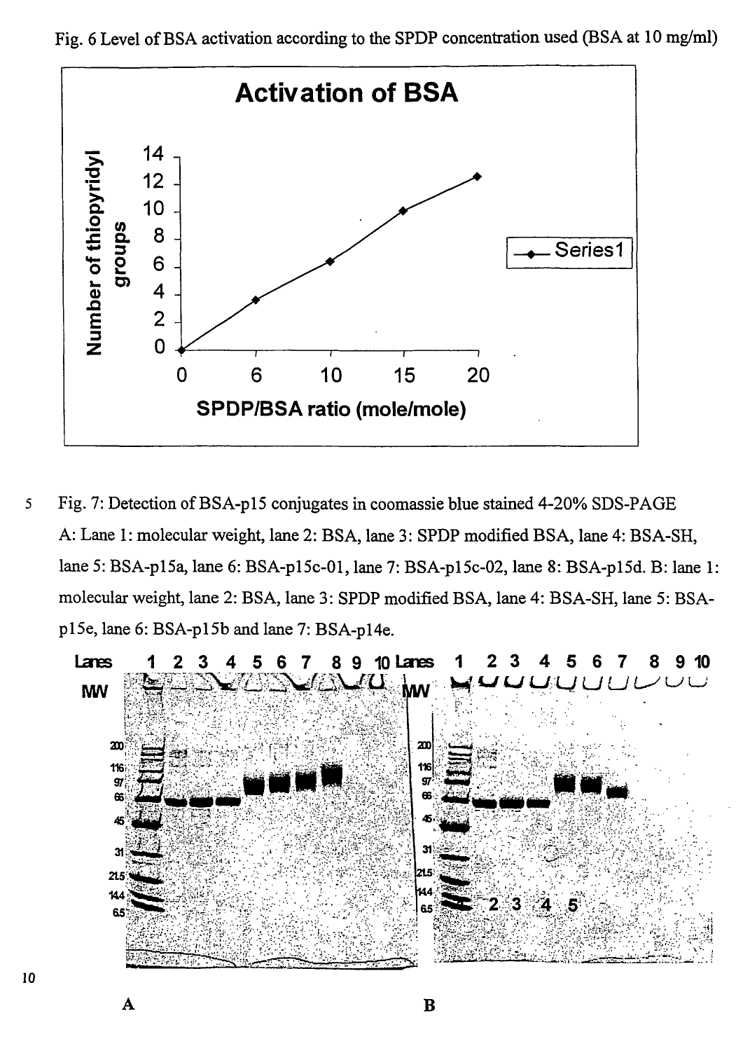

A maximum of 8 to 10 thiopyridyl groups can be added on BSA. A higher thiopyridyl number can be obtained if a 20 fold molar excess of SPDP over BSA was used (FIG. 6). However, a slight clouding was then observed during the reaction resulting in a lower yield of SPDP modified BSA.

Assays of reduction of pyridyl disulfide with DTT were carried out in sodium acetate pH 4.5 (to avoid reduction of native disulphide bonds) or in phosphate buffer (for SPDP modified PD). Efficacy of DTT was determined by release of pyridine-2-thione.

2. Conjugation of Constrained p15 Peptides

Five constrained peptides were conjugated to the BSA using the chemistry described hereabove:

| | |
|---|---|
| Original sequence: | |
| EDGQVMDVD | (SEQ ID NO. 1) |
| p15a: | |
| GGCLEDGQVMDVDC | (SEQ ID NO. 324) |
| p15b: | |
| Ac-CLEDGQVMDCGSK-NH₂ | (SEQ ID NO. 325) |
| p15c: | |
| Ac-CLEDGQVMDVDLCGSK-NH₂ | (SEQ ID NO. 326) |
| p15d: | |
| Ac-CLEDGQVMDVDLCPREAAEGDK-NH₂ | (SEQ ID NO. 327) |
| p15e: | |
| Ac-CLEDGQVMDVDLCGGSSGGK-NH₂ | (SEQ ID NO. 328) |

The resulting conjugates were soluble and were characterized by SDS-PAGE (Coomassie blue-staining) (FIG. 7).

3. Conjugation of Constrained p14 Peptides

Three constrained peptides were conjugated:

| | |
|---|---|
| Original sequence: | |
| PEWPGSRDKRT | (SEQ ID NO. 63) |
| p14e: | |
| ACPEWPGSRDRCTLAG-NH₂ | (SEQ ID NO. 323) |
| p14f: | |
| Ac-CPEWPGSRDRCGSK-NH₂ | (SEQ ID NO. 304) |
| p14i: | |
| Ac-CWPGSRDRRCGSK-NH₂ | (SEQ ID NO. 305) |

The resulting conjugates were soluble and were characterized by SDS-PAGE (coomassie blue-staining and western blot) (FIG. 7B, lane 7, FIG. 8 and FIG. 9).

4. Thiol-Disulfide Exchange

Compounds containing a disulfide group are able to participate in disulfide exchange reactions with another thiol. The disulfide exchange process involves attack of the thiol at the disulfide, breaking the S—S bond, with subsequent formation of a new mixed disulfide constituting a portion of the original disulfide compound. If the thiol is present in excess, the mixed disulfide can go on to form a symmetrical disulfide consisting entirely of the thiol reducing agent. If the thiol is not present in large excess, the mixed disulfide product is the end result.

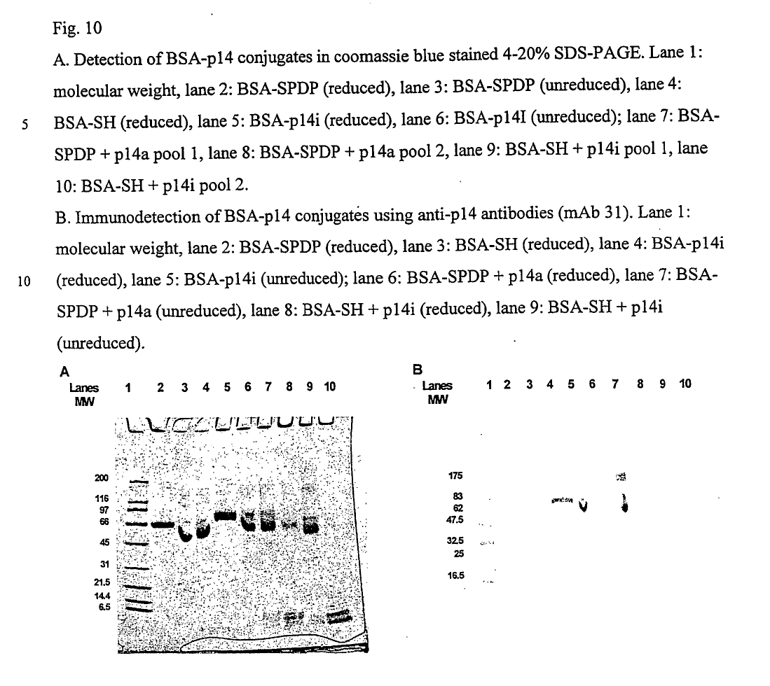

In order to test if a disulfide interchange could be observed during the reaction between BSA-SH and the maleimide activated disulfide bridge cyclised peptide, a reaction between BSA-SH and the unmodified p14i peptide was realized in the same coupling conditions (buffer, pH, ratio peptide/carrier and temperature). After 1 hour, the sample was dialyzed or applied on a desalting column (sephadex G25) equilibrated with phosphate buffer pH 6.8. The resulting product was analyzed on SDS-PAGE (coomassie blue staining) (FIG. 10). A positive control was included resulting from the reaction between SPDP-modified BSA and p14a peptide (AcAPEWPGSRDKRTLAGGC) in which disulfide interchange occurs (FIG. 3A). The resulting conjugate was purified by dialysis or by gel filtration.

No increase of the molecular size was seen for the product resulting of the reaction between BSA-SH and p14i (FIG. 10A: Lane 9). Moreover, no protein was detected with the mAb 31 (FIG. 1B: lane 9) suggesting the absence of disulfide interchange during the reaction at least in the conditions used for the coupling.

Conclusions

The combination of two chemistries was used to conjugate constrained peptides to a carrier. Soluble conjugates with 6 to 8 peptides on the carrier were obtained and were characterized by SDS-PAGE with antibodies against p14. The resulting conjugates were principally obtained by the reaction between the GMBS activated peptide and BSA-SH and not by disulfide interchange as confirmed by Western-blot. These results demonstrate that these chemistries can be used to conjugate constrained peptides to a carrier. In the above examples the maleimide was added to the peptide via reaction of maleimide-N-hydroxysuccinimide ester reagents with a lysine side-chain or with a N-terminal amino group. It is clear that alternative methods of adding the maleimide group can be readily conceived: notably for peptides containing a lysine within the epitope, the maleimide can be added during peptide synthesis prior to final deprotection of the side-chains and cleavage of the peptide.

### EXAMPLE 2

Immune Response Induced by Different Disulphide Bridged Peptide-BSA Conjugates.

To evaluate the immunogenicity of the conjugates produced in Example 1, 10 mice per group were immunised intramuscularly (IM) on days 0, 14 and 28 with 25 μg of conjugate mixed with AS2 adjuvant (oil/water emulsion, 3D-MPL, QS21). The serologic response for the P14 peptides was analysed by ELISA on days 28 and 42 (14 post III). The results are shown below in Table 4.

TABLE 4
IgG response against P14 peptides, day 14 post III

| Peptide | | | | | | | | | | | | | |
|---|---|---|---|---|---|---|---|---|---|---|---|---|---|
| conjugate | IgG anti-peptide responses (midpoint titre) | | | | | | | | | | Average. | st. deviation. | geomean |
| P14e | 3151 | 3224 | 3051 | | 2873 | 4647 | 1461 | 4227 | 3821 | 2345 | 3200 | 963 | 3051 |
| P14f | 67086 | 36031 | 74838 | 56496 | 51304 | 92885 | 92868 | 113041 | 89155 | 101502 | 77521 | 24519 | 73541 |
| P14I | 85882 | 39268 | 39460 | 57276 | 50834 | 54664 | 62263 | 36621 | 26202 | 28989 | 48146 | 17926 | 45336 |

Immune Response Induced by Different P15-BSA Conjugates.

The P15 peptide conjugates produced in Example 1 were also used to immunise 10 mice per group,intramuscularly (IM) on days 0, 14 and 28 with 25 μg of conjugate mixed with AS2 adjuvant (oil/water emulsion, 3D-MPL, QS21). Anti peptide and anti-IgE antibody responses are shown in Table 5 (14 days post III). Very homogenous responses were obtained with all cyclic P15 peptides. Anti-IgE antibody responses were assayed by comparison with a monoclonal antibody, mAb 11, which is known to recognise the P15 target site (c-d loop of Cε2) and inhibit histamine release in the Human Basophil Assay, the levels of anti-IgE were subsequently expressed as μg/ml mAb11 equivalents.

TABLE 5
Immune response by cyclic P15-BSA conjugates.

| | | | | anti-IgE (μg/ml or | | |
|---|---|---|---|---|---|---|
| BSA | anti-peptide (midpoint titre) | | | mAB11 equivalent) | | |
| conjugate | average | St Dev. | geomean | average | St Dev. | geomean |
| P15b | 11169 | 10766 | 8385 | 70 | 104 | 35 |
| P15c | 66452 | 10917 | 65685 | 200 | 64 | 189 |
| P15d | 35118 | 11601 | 32801 | 174 | 168 | 111 |
| P15e | 57432 | 16589 | 55207 | 129 | 68 | 113 |

Human Basophil Assays

Two types of assay were performed with human basophils (HBA), one to determine the anaphylactogenicity of the vaccine induced antibodies, consisting of adding the antibodies to isolated PBMC; and a second to measure the inhibition of Lol P I (a strong allergen) triggered histamine release by pre-incubation of the HBA with the vaccine induced antibodies.

Blood was collected by venepuncture from 4 allergic donors into tubes containing 0.1 volumes 2.7% EDTA, pH 7.0. It is then diluted 1/2 with an equal volume of HBH medium containing 0.1% human serum albumin (HBH/HSA). The resulting cell suspension was layered over 50% volume Ficoll-Paque and centrifuged at 400 g for 30 minutes at room temperature. The peripheral blood mononuclear cell (PBMC) layer at the interface is collected and the pellet is discarded. The cells are washed once in HBH/HSA, counted, and re-suspended in HBH/HSA at a cell density of 2.0×10⁶ per ml. 100 μl cell suspension are added to wells of a V-bottom 96-well plate containing 100 μl diluted test sample or vaccine induced antibody. Each test sample is tested at a range of dilutions with 6 wells for each dilution. Well contents are mixed briefly using a plate shaker, before incubation at 37° C. for 30 minutes with shaking at 120 rpm.

For each serum dilution 3 wells are triggered by addition of 10 μl Lol p I extract (final dilution 1/10000) and 3 wells have 10 μl HBH/HSA added for assessment of anaphylactogenicity. Well contents are again mixed briefly using a plate shaker, before incubation at 37° C. for a further 30 minutes with shaking at 120 rpm. Incubations are terminated by centrifugation at 500 g for 5 min. Supernatants are removed for histamine assay using a commercially available histamine EIA measuring kit (Immunotech). Control wells containing cells without test sample are routinely included to determine spontaneous and triggered release. Wells containing cells ±0.05% Igepal detergent are also included to determine total cell histarnine.

The results are expressed as following:

Anaphylactogenesis Assay

Histamine release due to test samples=% histamine release from test sample treated cells−% spontaneous histamine release.

Blocking Assay

The degree of inhibition of histamine release can be calculated using the formula:

% inhibition=1−(histamine release from test sample treated cells*)×100(histamine release from antigen stimulated cells*)

Values corrected for spontaneous release.

Results

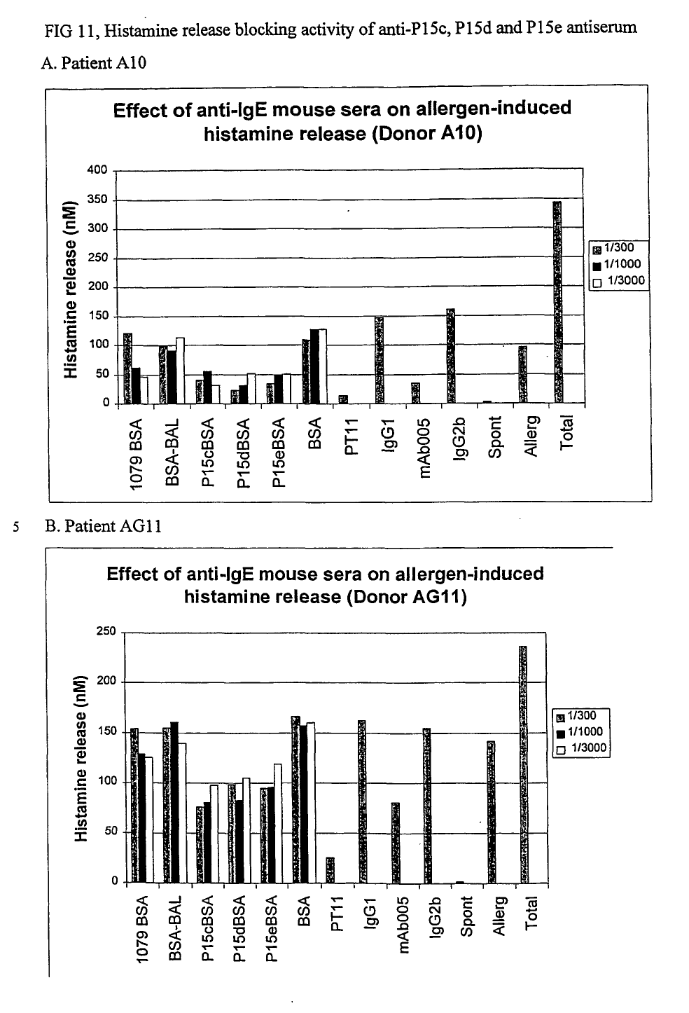

The results of the histamine release activity of the P15 disulphide bridge cyclised peptides conjugated to the BSA carriers using the chemistry of the present invention are shown in FIGS. 11 to 14.

FIGS. 11, A and B, show the histamine release blocking activity of antiserum induced by P15c, P15d and P15e; in comparison with the positive controls: 1079 BSA, PT11 and mAb005, and the negative controls BSA-BAL (activated carrier alone), anti-BSA, non-specific isotype controls (IgG1 and IgG2b); also shown are the data produced for spontanteous release of histamine, and histamine release after triggering with allergen, and total histamine content of the cells (released by detergent).

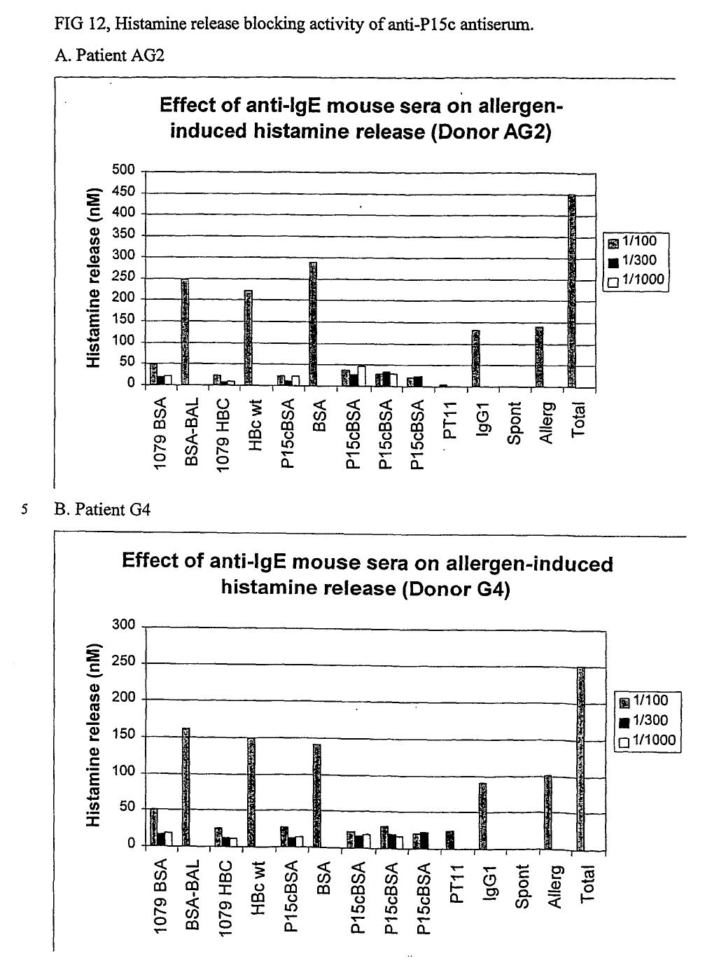

FIGS. 12, A and B, show the histamine release blocking activity of antiserum induced by P15c compared to the same controls as in FIG. 11, with the addition of a further positive control 1079 HBC, and one additional negative control HBC wt.

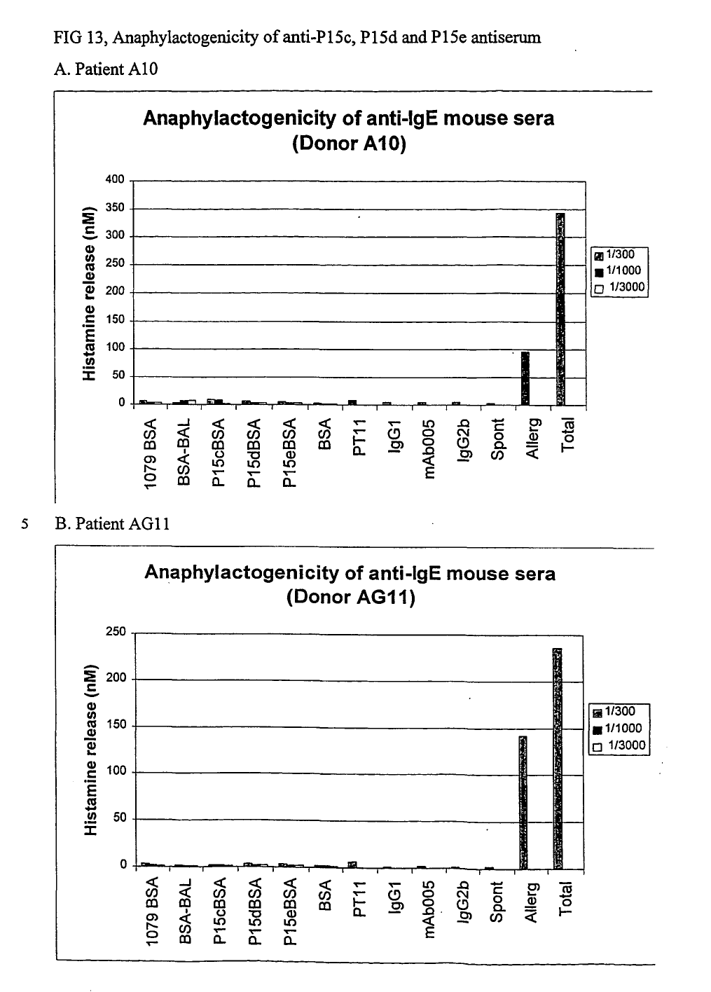

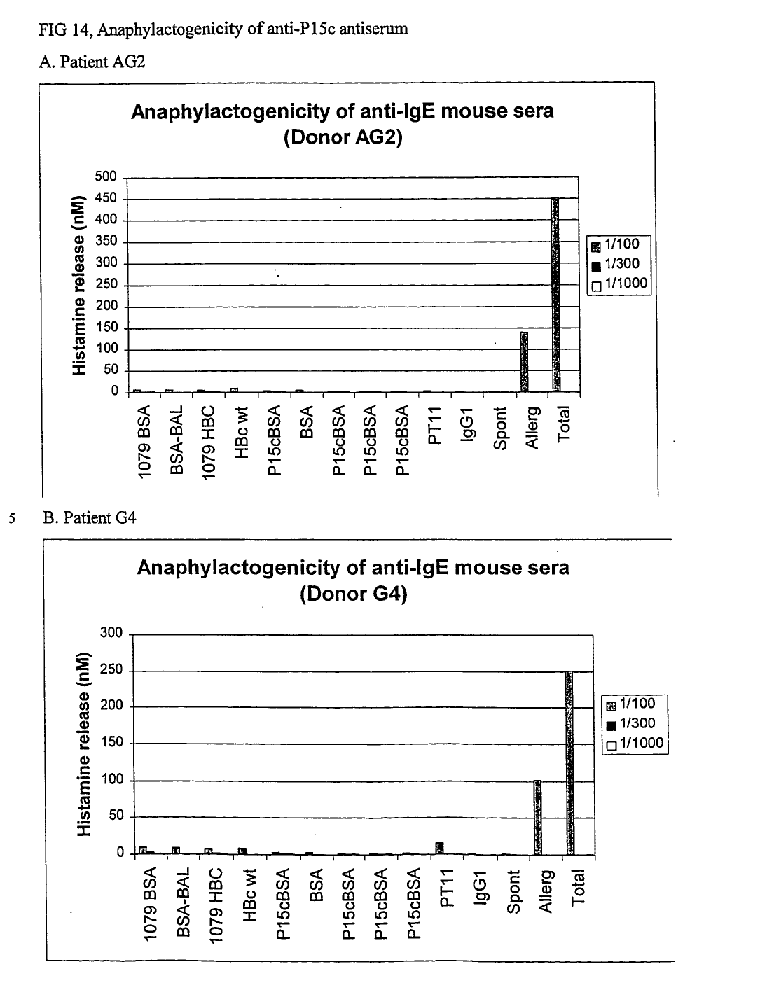

FIG. 13 shows the anaphylactogenicity of the same test samples (antiserum added to HBA in the absence of allergen) as described for FIG. 11 (P15c, P15d and P15e). FIG. 14 shows the anaphylactogenicity of the same test samples as described for FIG. 12.

In summary, P15c, P15d and P15e induced antisera that inhibited histamine release from human basophils after triggering with allergen, without the antiserum being anaphylactogenic themselves.

1. A process for the manufacture of a vaccine immunogen comprising conjugating a disulphide bridge cyclised peptide to an immunogenic carrier comprising, (a) adding to a disulphide cyclised peptide a moiety comprising a reactive group which is capable of forming thio-ether linkages with thiol bearing carriers, and (b) reacting the activated cyclised peptide thus formed with a thiol bearing immunogenic carrier.

2. A process as claimed in claim 1 wherein the reactive group capable of forming thio-ether linkages with thiol bearing carriers is a maleimide group.

3. A process as claimed in claim 1 wherein the disulphide bridge cyclised peptide is derived from human IgE.

4. A process as claimed in claim 3, wherein the human IgE peptide is selected from any one of SEQ ID NOs. 1 to 328.

5. A process as claimed in claim 1, wherein the carrier is selected from Haemophilus Influenzae Protein D, BSA, Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD).

6. A disulphide bridge cyclised IgE peptide maleimide derivative.

7. Use of a peptide derivative as claimed in claim 6, in the manufacture of a medicament for the treatment of allergy.

8. A conjugate suitable for use in a vaccine, of formula (I): 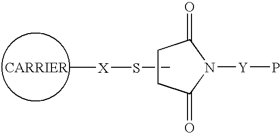
wherein, carrier is an immunogenic carrier molecule, X is either a linker or a bond, Y is either a linker or a bond, and P is a disulphide bridge cyclised peptide.

9. A conjugate as claimed in claim 8 wherein P is selected from the following group SEQ ID NO.s 99, 304, 305, 306, 307, 308, 309, 310, 311, 312, 313, 314, 315, 316, 317, 318, 319, 320, 321, 322, 323, 324, 325, 326, 327, and 328.

10. A vaccine composition comprising the product of the process claimed in any one of claims 1 to 5, and a suitable adjuvant or carrier.

11. A vaccine composition comprising a conjugate as claimed in claim 8 or 9, and a suitable adjuvant or carrier.

12. A vaccine as claimed in claim 10 or 11, wherein the vaccine is an allergy vaccine.

13. A conjugate as claimed in claim 8 for the treatment of allergy.


SEQUENCE LISTING (328 sequences)

SEQ ID NO: 1 - protein, 9 aa, Homo sapiens
EDGQVMDVD

SEQ ID NO: 2 - protein, 8 aa, Homo sapiens
STTQEGEL

SEQ ID NO: 3 - protein, 10 aa, Homo sapiens
SQKHWLSDRT

SEQ ID NO: 4 - protein, 10 aa, Homo sapiens
GHTFEDSTKK

SEQ ID NO: 5 - protein, 8 aa, Homo sapiens
GGGHFPPT

SEQ ID NO: 6 - protein, 6 aa, Homo sapiens
PGTINI

SEQ ID NO: 7 - protein, 5 aa, Homo sapiens
FTPPT

SEQ ID NO: 8 - protein, 13 aa, Homo sapiens
CLEDGQVMDVDLL

SEQ ID NO: 9 - protein, 13 aa, Homo sapiens
LLDVDMVQGDELC

SEQ ID NO: 10 - protein, 13 aa, Homo sapiens
WLEDGQVMDVDLC

SEQ ID NO: 11 - protein, 7 aa, Homo sapiens
QVMDVDL

SEQ ID NO: 12 - protein, 10 aa, Homo sapiens
LEDGQVMDVD

SEQ ID NO: 13 - protein, 10 aa, Homo sapiens
CSTTQEGELA

SEQ ID NO: 14 - protein, 6 aa, Homo sapiens
TTQEGE

SEQ ID NO: 15 - protein, 11 aa, Homo sapiens
CSQKHWLSDRT

SEQ ID NO: 16 - protein, 22 aa, Homo sapiens
TYQGHTFEDSTKKCADSNPRGV

SEQ ID NO: 17 - protein, 6 aa, Homo sapiens
GGHFPP

SEQ ID NO: 18 - protein, 17 aa, Homo sapiens
CCVADPETQMTPSSEMF

SEQ ID NO: 19 - protein, 17 aa, Homo sapiens
CCVADPETQMTPSSEMF

SEQ ID NO: 20 - protein, 17 aa, Homo sapiens
CCVTDVQTTNMDVPAGQ

SEQ ID NO: 21 - protein, 17 aa, Homo sapiens
TCCVTDIPPPDYEQSLG

SEQ ID NO: 22 - protein, 17 aa, Homo sapiens
CCESDIPLNELHALADP

SEQ ID NO: 23 - protein, 17 aa, Homo sapiens
CCKSDIPSPVTQFNTMK

SEQ ID NO: 24 - protein, 17 aa, Homo sapiens
CCQSDVPHQPGINDLHV

SEQ ID NO: 25 - protein, 17 aa, Homo sapiens
CCMSDTPDISRLPVPDS

SEQ ID NO: 26 - protein, 17 aa, Homo sapiens
CCMSDSPADPNRGLPIW

SEQ ID NO: 27 - protein, 14 aa, Homo sapiens
CCLSDDAPTLPVRR

SEQ ID NO: 28 - protein, 17 aa, Homo sapiens
CCITDVPQGVMYKGSPD

SEQ ID NO: 29 - protein, 17 aa, Homo sapiens
ECKVDGQLSDSPLLRNN

SEQ ID NO: 30 - protein, 17 aa, Homo sapiens
CCMTDDPMDPNSTWAIR

SEQ ID NO: 31 - protein, 17 aa, Homo sapiens
CCMTDDPMYTNSTWAIR

SEQ ID NO: 32 - protein, 17 aa, Homo sapiens
CCVDDTPNSGLAMRVSK

SEQ ID NO: 33 - protein, 17 aa, Homo sapiens
CCEVDDFPTHHPGWTLR

SEQ ID NO: 34 - protein, 17 aa, Homo sapiens
SCNLNHQSCDIPPVKQI

SEQ ID NO: 35 - protein, 17 aa, Homo sapiens
CCMADQELDLGHNAANA

SEQ ID NO: 36 - protein, 12 aa, Homo sapiens
CCVMDLELASGF

SEQ ID NO: 37 - protein, 12 aa, Homo sapiens
CCVMDIEVRGSA

SEQ ID NO: 38 - protein, 12 aa, Homo sapiens
CCQRDVELVFGS

SEQ ID NO: 39 - protein, 12 aa, Homo sapiens
CCRADFEVGNGG

SEQ ID NO: 40 - protein, 12 aa, Homo sapiens
CCVSDEPAGVRD

SEQ ID NO: 41 - protein, 12 aa, Homo sapiens
GAGWQEKDKELR

SEQ ID NO: 42 - protein, 12 aa, Homo sapiens
GAMTAGQLSDLP

SEQ ID NO: 43 - protein, 12 aa, Homo sapiens
VAGGQVVDRELK

SEQ ID NO: 44 - protein, 12 aa, Homo sapiens
KAGEQAMDMELR

SEQ ID NO: 45 - protein, 11 aa, Homo sapiens
RGRNQIMDLEI

SEQ ID NO: 46 - protein, 11 aa, Homo sapiens
QIDRQITDTLL

SEQ ID NO: 47 - protein, 11 aa, Homo sapiens
REQQISDVPRV

SEQ ID NO: 48 - protein, 12 aa, Homo sapiens
CQAMDAEILNQV

SEQ ID NO: 49 - protein, 11 aa, Homo sapiens
GQMMDTELLNR

SEQ ID NO: 50 - protein, 11 aa, Homo sapiens
SMEGQVRDIQV

SEQ ID NO: 51 - protein, 11 aa, Homo sapiens
YQQRDLELLAE

SEQ ID NO: 52 - protein, 11 aa, Homo sapiens
SMGQKVDRELV

SEQ ID NO: 53 - protein, 11 aa, Homo sapiens
SMGQEVDRELV

SEQ ID NO: 54 - protein, 11 aa, Homo sapiens
AENDQMVDWEI

SEQ ID NO: 55 - protein, 11 aa, Homo sapiens
GGWQESDIPGR

SEQ ID NO: 56 - protein, 11 aa, Homo sapiens
GGWQEKDKELR

SEQ ID NO: 57 - protein, 12 aa, Homo sapiens
HCCRIDREVSGA

SEQ ID NO: 58 - protein, 17 aa, Homo sapiens
DCDWINPPDPPHFWKDT

SEQ ID NO: 59 - protein, 12 aa, Homo sapiens
DALDERAWRARA

SEQ ID NO: 60 - protein, 22 aa, Homo sapiens
RASGKPVNHSTRKEEKQRNGTL

SEQ ID NO: 61 - protein, 9 aa, Homo sapiens
GTRDWIEGE

SEQ ID NO: 62 - protein, 19 aa, Homo sapiens
PHLPRALMRSTTKTSGPRA

SEQ ID NO: 63 - protein, 11 aa, Homo sapiens
PEWPGSRDKRT

SEQ ID NO: 64 - protein, 5 aa, Homo sapiens
EQKDE

SEQ ID NO: 65 - protein, 19 aa, Homo sapiens
LSRPSPFDLFIRKSPTITC

SEQ ID NO: 66 - protein, 21 aa, Homo sapiens
WLHNEVQLPDARHSTTQPRKT

SEQ ID NO: 67 - protein, 23 aa, Homo sapiens
CRASGKPVNHSTRKEEKQRNGLL

SEQ ID NO: 68 - protein, 11 aa, Homo sapiens
GKPVNHSTGGC

SEQ ID NO: 69 - protein, 18 aa, Homo sapiens
GKPVNHSTRKEEKQRNGC

SEQ ID NO: 70 - protein, 20 aa, Homo sapiens
CGKPVNHSTRKEEKQRNGLL

SEQ ID NO: 71 - protein, 14 aa, Homo sapiens
RASGKPVNHSTGGC

SEQ ID NO: 72 - protein, 11 aa, Homo sapiens
CGTRDWIEGLL

SEQ ID NO: 73 - protein, 12 aa, Homo sapiens
CGTRDWIEGETL

SEQ ID NO: 74 - protein, 12 aa, Homo sapiens
GTRDWIEGETGC

SEQ ID NO: 75 - protein, 12 aa, Homo sapiens
CHPHLPRALMLL

SEQ ID NO: 76 - protein, 12 aa, Homo sapiens
CGTHPHLPRALM

SEQ ID NO: 77 - protein, 13 aa, Homo sapiens
THPHLPRALMRSC

SEQ ID NO: 78 - protein, 14 aa, Homo sapiens
GPHLPRALMRSSSC

SEQ ID NO: 79 - protein, 13 aa, Homo sapiens
APEWPGSRDKRTC

SEQ ID NO: 80 - protein, 17 aa, Homo sapiens
APEWPGSRDKRTLAGGC

SEQ ID NO: 81 - protein, 17 aa, Homo sapiens
CGGATPEWPGSRDKRTL

SEQ ID NO: 82 - protein, 13 aa, Homo sapiens
CTRKDRSGPWEPA

SEQ ID NO: 83 - protein, 11 aa, Homo sapiens
CGAEWEQKDEL

SEQ ID NO: 84 - protein, 11 aa, Homo sapiens
AEWEQKDEFIC

SEQ ID NO: 85 - protein, 9 aa, Homo sapiens
GEQKDEFIC

SEQ ID NO: 86 - protein, 10 aa, Homo sapiens
CAEGEQKDEL

SEQ ID NO: 87 - protein, 6 aa, Homo sapiens
LFIRKS

SEQ ID NO: 88 - protein, 7 aa, Homo sapiens
PSKGTVN

SEQ ID NO: 89 - protein, 23 aa, Homo sapiens
LHNEVQLPDARHSTTQPRKTKGS

SEQ ID NO: 90 - protein, 6 aa, Homo sapiens
SVNPGK

SEQ ID NO: 91 - protein, 13 aa, Homo sapiens
CPEWPGCRDKRTG

SEQ ID NO: 92 - protein, 13 aa, Homo sapiens
TPEWPGCRDKRCG

SEQ ID NO: 93 - protein, 14 aa, Homo sapiens
DPEWPGSRDKKGSC

SEQ ID NO: 94 - protein, 13 aa, Homo sapiens
DWPGSRDKRKGSC

SEQ ID NO: 95 - protein, 19 aa, Homo sapiens
DATPEWPGSRDKRTLKGSC

SEQ ID NO: 96 - protein, 13 aa, Artificial Sequence
  Artificial variant of Homo sapiens IgE peptide
CLEDGQVMDVDLC

SEQ ID NO: 97 - protein, 16 aa, Artificial Sequence
  Artificial variant of Homo sapiens IgE peptide
CFINKQMADLELCPRE

SEQ ID NO: 98 - protein, 16 aa, Artificial Sequence
  Artificial variant of Homo sapiens IgE peptide
CFMNKQLADLELCPRE

SEQ ID NO: 99 - protein, 22 aa, Artificial Sequence
  Artificial variant of Homo sapiens IgE peptide
CLEDGQVMDVDLCPREAAEGDK

SEQ ID NO: 100 - protein, 20 aa, Artificial Sequence
  Artificial variant of Homo sapiens IgE peptide
CLEDGQVMDVDLCGGSSGGP

SEQ ID NO: 101 - protein, 21 aa, Artificial Sequence
  Artificial variant of Homo sapiens IgE peptide
CLEDGQVMDVDCPREAAEGDK

SEQ ID NO: 102 - protein, 17 aa, Artificial Sequence
  Artificial variant of Homo sapiens IgE peptide
KCREVWLGESETIMDCE

SEQ ID NO: 103 - protein, 17 aa, Artificial Sequence
  Artificial variant of Homo sapiens IgE peptide
ACREVWLGESETIMDCD

SEQ ID NO: 104 - protein, 17 aa, Artificial Sequence
  Artificial variant of Homo sapiens IgE peptide
SCREVWLGESETVMDCG

SEQ ID NO: 105 - protein, 17 aa, Artificial Sequence
  Artificial variant of Homo sapiens IgE peptide
NCQDLMLREDAGCWSKM

SEQ ID NO: 106 - protein, 17 aa, Artificial Sequence
  Artificial variant of Homo sapiens IgE peptide
DCEEPMCSPVLLQQLKL

SEQ ID NO: 107 - protein, 13 aa, Artificial Sequence
  Artificial variant of Homo sapiens IgE peptide
CFINKQMADLELC

SEQ ID NO: 108 - protein, 13 aa, Artificial Sequence
  Artificial variant of Homo sapiens IgE peptide
CFMNKQLADLELC

SEQ ID NO: 109 - protein, 16 aa, Artificial Sequence
  Artificial variant of Homo sapiens IgE peptide
KCREVWLGESETIMDC

SEQ ID NO: 110 - protein, 17 aa, Artificial Sequence
  Artificial variant of Homo sapiens IgE peptide
HCQQVFFPQDYLWCQRG

SEQ ID NO: 111 - protein, 17 aa, Artificial Sequence
  Artificial variant of Homo sapiens IgE peptide
SCREVWLGGSEMIMDCE

SEQ ID NO: 112 - protein, 17 aa, Artificial Sequence
  Artificial variant of Homo sapiens IgE peptide
ECNQNLSGSLRHVDLNC

SEQ ID NO: 113 - protein, 17 aa, Artificial Sequence
  Artificial variant of Homo sapiens IgE peptide
DCEEPMCSPVLLQKLKP

SEQ ID NO: 114 - protein, 17 aa, Artificial Sequence
  Artificial variant of Homo sapiens IgE peptide
SCREVWLGGSEMIMDCE

SEQ ID NO: 115 - protein, 17 aa, Artificial Sequence
  Artificial variant of Homo sapiens IgE peptide
RCDQQLPRDSYTFCMMS

SEQ ID NO: 116 - protein, 17 aa, Artificial Sequence
  Artificial variant of Homo sapiens IgE peptide
SCPAFPREGDLCAPPTV

SEQ ID NO: 117 - protein, 17 aa, Artificial Sequence
  Artificial variant of Homo sapiens IgE peptide
FCPEPICSPPLSRMTLS

SEQ ID NO: 118 - protein, 12 aa, Artificial Sequence
  Artificial variant of Homo sapiens IgE peptide
VCDECVSRELAL

SEQ ID NO: 119 - protein, 12 aa, Artificial Sequence
  Artificial variant of Homo sapiens IgE peptide
WCLEPECAPGLL

SEQ ID NO: 120 - protein, 12 aa, Artificial Sequence
  Artificial variant of Homo sapiens IgE peptide
VCDECVSRELAL

SEQ ID NO: 121 - protein, 12 aa, Artificial Sequence
  Artificial variant of Homo sapiens IgE peptide
DCLSKGQMADLC

SEQ ID NO: 122 - protein, 12 aa, Artificial Sequence
  Artificial variant of Homo sapiens IgE peptide
SCQGREVRRECW

SEQ ID NO: 123 - protein, 17 aa, Artificial Sequence
  Artificial variant of Homo sapiens IgE peptide
WCREVWLGESETIMDCE

SEQ ID NO: 124 - protein, 17 aa, Artificial Sequence
  Artificial variant of Homo sapiens IgE peptide
ACREVWLGESETIMDCD

SEQ ID NO: 125 - protein, 17 aa, Artificial Sequence
  Artificial variant of Homo sapiens IgE peptide
GCAEPKCWQALHQKLKP

SEQ ID NO: 126 - protein, 17 aa, Artificial Sequence
  Artificial variant of Homo sapiens IgE peptide
ECRGPNMQMQDHCPTTD

SEQ ID NO: 127 - protein, 17 aa, Artificial Sequence
  Artificial variant of Homo sapiens IgE peptide
QCNAVLEGLQMVDHCWN

SEQ ID NO: 128 - protein, 17 aa, Artificial Sequence
  Artificial variant of Homo sapiens IgE peptide
HCKNEFKKGQWTYSCSD

SEQ ID NO: 129 - protein, 17 aa, Artificial Sequence
  Artificial variant of Homo sapiens IgE peptide
QCRQFVMNQSEKEFGQC

SEQ ID NO: 130 - protein, 17 aa, Artificial Sequence
  Artificial variant of Homo sapiens IgE peptide
NCFMNKQLADLELCPRE

SEQ ID NO: 131 - protein, 17 aa, Artificial Sequence
  Artificial variant of Homo sapiens IgE peptide
SCAYTAQRQCSDVPNPG

SEQ ID NO: 132 - protein, 19 aa, Artificial Sequence
  Artificial variant of Homo sapiens IgE peptide
GCFMNKQMADLELCPRTAA

SEQ ID NO: 133 - protein, 19 aa, Artificial Sequence
  Artificial variant of Homo sapiens IgE peptide
ACFMNKQMADLELCPRVAA

SEQ ID NO: 134 - protein, 19 aa, Artificial Sequence
  Artificial variant of Homo sapiens IgE peptide
GCFINKQLADLELCPRVAA

SEQ ID NO: 135 - protein, 19 aa, Artificial Sequence
  Artificial variant of Homo sapiens IgE peptide
GCFMNKQLADWELCPRAAA

SEQ ID NO: 136 - protein, 19 aa, Artificial Sequence
  Artificial variant of Homo sapiens IgE peptide
ECFMNKQLADSELCPRVAA

SEQ ID NO: 137 - protein, 19 aa, Artificial Sequence
  Artificial variant of Homo sapiens IgE peptide
GCFMNKQLADPELCPREAE

SEQ ID NO: 138 - protein, 19 aa, Artificial Sequence
  Artificial variant of Homo sapiens IgE peptide
GCFMNKQLVDLELCPRGAA

SEQ ID NO: 139 - protein, 19 aa, Artificial Sequence
  Artificial variant of Homo sapiens IgE peptide
GCFMNKQLADLELCPREAA

SEQ ID NO: 140 - protein, 19 aa, Artificial Sequence
  Artificial variant of Homo sapiens IgE peptide
GCFMNKQQADLELCPRGAA

SEQ ID NO: 141 - protein, 19 aa, Artificial Sequence
  Artificial variant of Homo sapiens IgE peptide
GCFINKQMADLELCPREAA

SEQ ID NO: 142 - protein, 20 aa, Artificial Sequence
  Artificial variant of Homo sapiens IgE peptide
CLEDGQVMDVDCPREAAEGD

SEQ ID NO: 143 - protein, 21 aa, Artificial Sequence
  Artificial variant of Homo sapiens IgE peptide
CLEDGQVMDVDLCPREAAEGD

SEQ ID NO: 144 - protein, 17 aa, Artificial Sequence
  Artificial variant of Homo sapiens IgE peptide
QCNAVLEGLQMVDHCWN

SEQ ID NO: 145 - protein, 17 aa, Artificial Sequence
  Artificial variant of Homo sapiens IgE peptide
ECLKIEQQCADIVEIPR

SEQ ID NO: 146 - protein, 17 aa, Artificial Sequence
  Artificial variant of Homo sapiens IgE peptide
SCAYTAQRQCSDVPNPG

SEQ ID NO: 147 - protein, 17 aa, Artificial Sequence
  Artificial variant of Homo sapiens IgE peptide
ECRGPNMQMQDHCPTTD

SEQ ID NO: 148 - protein, 17 aa, Artificial Sequence
  Artificial variant of Homo sapiens IgE peptide
ECLVYGQMADCAAGGWP

SEQ ID NO: 149 - protein, 17 aa, Artificial Sequence
  Artificial variant of Homo sapiens IgE peptide
QCRQFVMNQSEKEFGQC

SEQ ID NO: 150 - protein, 17 aa, Artificial Sequence
  Artificial variant of Homo sapiens IgE peptide
HCKNEFKKGQWTYSCSD

SEQ ID NO: 151 - protein, 12 aa, Artificial Sequence
  Artificial variant of Homo sapiens IgE peptide
CAPGMGCWESVK

SEQ ID NO: 152 - protein, 17 aa, Artificial Sequence
  Artificial variant of Homo sapiens IgE peptide
SCREVWLGGSEMIMDCE

SEQ ID NO: 153 - protein, 17 aa, Artificial Sequence
  Artificial variant of Homo sapiens IgE peptide
SCPAFPREGDLCAPPTV

SEQ ID NO: 154 - protein, 17 aa, Artificial Sequence
  Artificial variant of Homo sapiens IgE peptide
FCPEPICSPPLSRMTLS

SEQ ID NO: 155 - protein, 17 aa, Artificial Sequence
  Artificial variant of Homo sapiens IgE peptide
ECNQNLSGSLRHVDLNC

SEQ ID NO: 156 - protein, 17 aa, Artificial Sequence
  Artificial variant of Homo sapiens IgE peptide
RCDQQLPRDSYTFCMMS

SEQ ID NO: 157 - protein, 17 aa, Artificial Sequence
  Artificial variant of Homo sapiens IgE peptide
HCQQVFFPQDYLWCQRG

SEQ ID NO: 158 - protein, 17 aa, Artificial Sequence
  Artificial variant of Homo sapiens IgE peptide
DCEEPMCSPVLLQKLKP

SEQ ID NO: 159 - protein, 17 aa, Artificial Sequence
  Artificial variant of Homo sapiens IgE peptide
NCQDQMLREDAGCWSKI

SEQ ID NO: 160 - protein, 17 aa, Artificial Sequence
  Artificial variant of Homo sapiens IgE peptide
HCEEPEYSPATRVFCGR

SEQ ID NO: 161 - protein, 17 aa, Artificial Sequence
  Artificial variant of Homo sapiens IgE peptide
ACFSRNGQVTDVPHSCY

SEQ ID NO: 162 - protein, 17 aa, Artificial Sequence
  Artificial variant of Homo sapiens IgE peptide
KCPTYPKPNDRCLWPVP

SEQ ID NO: 163 - protein, 17 aa, Artificial Sequence
  Artificial variant of Homo sapiens IgE peptide
YCPKYPLEGDCLLDNDY

SEQ ID NO: 164 - protein, 17 aa, Artificial Sequence
  Artificial variant of Homo sapiens IgE peptide
RCEEWLCIPPAPAFAPP

SEQ ID NO: 165 - protein, 17 aa, Artificial Sequence
  Artificial variant of Homo sapiens IgE peptide
TCGQSELRCASLETHHV

SEQ ID NO: 166 - protein, 16 aa, Artificial Sequence
  Artificial variant of Homo sapiens IgE peptide
NCNDNPMLDCMPAWSS

SEQ ID NO: 167 - protein, 12 aa, Artificial Sequence
  Artificial variant of Homo sapiens IgE peptide
SCQGREVRRECW

SEQ ID NO: 168 - protein, 12 aa, Artificial Sequence
  Artificial variant of Homo sapiens IgE peptide
VCDECVSRELAL

SEQ ID NO: 169 - protein, 12 aa, Artificial Sequence
  Artificial variant of Homo sapiens IgE peptide
WCLEPECAPGLL

SEQ ID NO: 170 - protein, 12 aa, Artificial Sequence
  Artificial variant of Homo sapiens IgE peptide
DCLSKGQMADLC

SEQ ID NO: 171 - protein, 12 aa, Artificial Sequence
  Artificial variant of Homo sapiens IgE peptide
VCDECVSRELAL

SEQ ID NO: 172 - protein, 12 aa, Artificial Sequence
  Artificial variant of Homo sapiens IgE peptide
GCPTWPRVGDHC

SEQ ID NO: 173 - protein, 12 aa, Artificial Sequence
  Artificial variant of Homo sapiens IgE peptide
RCQSARVVPECW

SEQ ID NO: 174 - protein, 12 aa, Artificial Sequence
  Artificial variant of Homo sapiens IgE peptide
SCAPSGDCGYKG

SEQ ID NO: 175 - protein, 12 aa, Artificial Sequence
  Artificial variant of Homo sapiens IgE peptide
GCPMWPQPDDEC

SEQ ID NO: 176 - protein, 12 aa, Artificial Sequence
  Artificial variant of Homo sapiens IgE peptide
ECPRWPLMGDGC

SEQ ID NO: 177 - protein, 12 aa, Artificial Sequence
  Artificial variant of Homo sapiens IgE peptide
GCQVGELVWCRE

SEQ ID NO: 178 - protein, 12 aa, Artificial Sequence
  Artificial variant of Homo sapiens IgE peptide
QCVRDGTRKVCM

SEQ ID NO: 179 - protein, 12 aa, Artificial Sequence
  Artificial variant of Homo sapiens IgE peptide
TCLVDRQESDVC

SEQ ID NO: 180 - protein, 12 aa, Artificial Sequence
  Artificial variant of Homo sapiens IgE peptide
DCVVDGDRLVCL

SEQ ID NO: 181 - protein, 12 aa, Artificial Sequence
  Artificial variant of Homo sapiens IgE peptide
RCEQGALRCVGE

SEQ ID NO: 182 - protein, 12 aa, Artificial Sequence
  Artificial variant of Homo sapiens IgE peptide
VCPPGWKNLGCN

SEQ ID NO: 183 - protein, 12 aa, Artificial Sequence
  Artificial variant of Homo sapiens IgE peptide
MCQGWEIVSECW

SEQ ID NO: 184 - protein, 25 aa, Artificial Sequence
  Artificial variant of Homo sapiens IgE peptide
ADGAGCFMNKQMADLELCPREAAEA

SEQ ID NO: 185 - protein, 25 aa, Artificial Sequence
  Artificial variant of Homo sapiens IgE peptide
ADGAGCFMNKQMADLELCPRTAAEA

SEQ ID NO: 186 - protein, 25 aa, Artificial Sequence
  Artificial variant of Homo sapiens IgE peptide
ADGAACFMNKQMADLELCPRVAAEA

SEQ ID NO: 187 - protein, 25 aa, Artificial Sequence
  Artificial variant of Homo sapiens IgE peptide
ADGAGCFINKQLADLELCPRVAAEA

SEQ ID NO: 188 - protein, 25 aa, Artificial Sequence
  Artificial variant of Homo sapiens IgE peptide
ADGAGCFINKQLADLELCPREAAEA

SEQ ID NO: 189 - protein, 25 aa, Artificial Sequence
  Artificial variant of Homo sapiens IgE peptide
ADGAGCFMNKQLADLEMCPRDDAEA

SEQ ID NO: 190 - protein, 25 aa, Artificial Sequence
  Artificial variant of Homo sapiens IgE peptide
ADGAGCFMNKQLADPELCPREAEEA

SEQ ID NO: 191 - protein, 25 aa, Artificial Sequence
  Artificial variant of Homo sapiens IgE peptide
ADGAGCFMNKQLVDLELCPRGAAEA

SEQ ID NO: 192 - protein, 25 aa, Artificial Sequence
  Artificial variant of Homo sapiens IgE peptide
ADGAGCFMNNQLADWELCPRAAAEA

SEQ ID NO: 193 - protein, 25 aa, Artificial Sequence
  Artificial variant of Homo sapiens IgE peptide
ADGAGCFMNKQMADWEMCPRAAAEA

SEQ ID NO: 194 - protein, 25 aa, Artificial Sequence
  Artificial variant of Homo sapiens IgE peptide
ADGAGCFMNKQQADLELCPRGAAEA

SEQ ID NO: 195 - protein, 25 aa, Artificial Sequence
  Artificial variant of Homo sapiens IgE peptide
ADGAECFMNKQLADSELCPRVAAEA

SEQ ID NO: 196 - protein, 25 aa, Artificial Sequence
  Artificial variant of Homo sapiens IgE peptide
ADGAGCFMNKQLADLELCPREAAEA

SEQ ID NO: 197 - protein, 25 aa, Artificial Sequence
  Artificial variant of Homo sapiens IgE peptide
ADGAGCFINMQMADQELCPRAAAEA

SEQ ID NO: 198 - protein, 25 aa, Artificial Sequence
  Artificial variant of Homo sapiens IgE peptide
ADGAGCFINKQMSDFELCPREAGEA

SEQ ID NO: 199 - protein, 25 aa, Artificial Sequence
  Artificial variant of Homo sapiens IgE peptide
ADGAGCFINKQMADLELCTREAAEA

SEQ ID NO: 200 - protein, 25 aa, Artificial Sequence
  Artificial variant of Homo sapiens IgE peptide
ADGAGCFINKQMADLELCPRQAAEA

SEQ ID NO: 201 - protein, 25 aa, Artificial Sequence
  Artificial variant of Homo sapiens IgE peptide
ADGAGCFINNQMADLELCPRGGAEA

SEQ ID NO: 202 - protein, 25 aa, Artificial Sequence
  Artificial variant of Homo sapiens IgE peptide
ADGAGCFINKQMADWELCPREGAEA

SEQ ID NO: 203 - protein, 25 aa, Artificial Sequence
  Artificial variant of Homo sapiens IgE peptide
ADGAGCFINKQMADLELCPSQAAEA

SEQ ID NO: 204 - protein, 25 aa, Artificial Sequence
  Artificial variant of Homo sapiens IgE peptide
ADGAGCFINKQMADLELCPREGAEA

SEQ ID NO: 205 - protein, 25 aa, Artificial Sequence
  Artificial variant of Homo sapiens IgE peptide
ADGAGCFINKQMADSELCPREPAEA

SEQ ID NO: 206 - protein, 25 aa, Artificial Sequence
  Artificial variant of Homo sapiens IgE peptide
ADGAGCFIKKQMADLELCPREAWEA

SEQ ID NO: 207 - protein, 25 aa, Artificial Sequence
  Artificial variant of Homo sapiens IgE peptide
ADGAECFINKQMADRELCAREVAEA

SEQ ID NO: 208 - protein, 25 aa, Artificial Sequence
  Artificial variant of Homo sapiens IgE peptide
ADGAGCFIDKQMADLELCPRAAAEA

SEQ ID NO: 209 - protein, 25 aa, Artificial Sequence
  Artificial variant of Homo sapiens IgE peptide
ADGAGCFINKQMADLELCRREAGEA

SEQ ID NO: 210 - protein, 25 aa, Artificial Sequence
  Artificial variant of Homo sapiens IgE peptide
ADGAGCFKNKQMVDSELCARQAAEA

SEQ ID NO: 211 - protein, 25 aa, Artificial Sequence
  Artificial variant of Homo sapiens IgE peptide
ADGAGCFQNKQMADLELCPREAAEA

SEQ ID NO: 212 - protein, 25 aa, Artificial Sequence
  Artificial variant of Homo sapiens IgE peptide
ADGAECFINKQRADLELCPGEAAEA

SEQ ID NO: 213 - protein, 25 aa, Artificial Sequence
  Artificial variant of Homo sapiens IgE peptide
ADGAGCFINKQMADSELCPAAAAEA

SEQ ID NO: 214 - protein, 25 aa, Artificial Sequence
  Artificial variant of Homo sapiens IgE peptide
ADGAGCFINRQMADPELCPREAAEA

SEQ ID NO: 215 - protein, 25 aa, Artificial Sequence
  Artificial variant of Homo sapiens IgE peptide
ADGAGCFIEKQMADMELCQARAAEA

SEQ ID NO: 216 - protein, 25 aa, Artificial Sequence
  Artificial variant of Homo sapiens IgE peptide
ADGAGCFINKQMADWELCPREAAEA

SEQ ID NO: 217 - protein, 25 aa, Artificial Sequence
  Artificial variant of Homo sapiens IgE peptide
ADGAGCFINNQMADLELCPREAAEA

SEQ ID NO: 218 - protein, 25 aa, Artificial Sequence
  Artificial variant of Homo sapiens IgE peptide
ADGAGCFIEKQMADMELCQRETAEA

SEQ ID NO: 219 - protein, 25 aa, Artificial Sequence
  Artificial variant of Homo sapiens IgE peptide
ADGAGCFINKQMADMELCPREAAEA

SEQ ID NO: 220 - protein, 25 aa, Artificial Sequence
  Artificial variant of Homo sapiens IgE peptide
ADGAGCFINKQMADLELCPREAAEA

SEQ ID NO: 221 - protein, 25 aa, Artificial Sequence
  Artificial variant of Homo sapiens IgE peptide
ADGAGCFRNKQMADLELCPREAAEA

SEQ ID NO: 222 - protein, 25 aa, Artificial Sequence
  Artificial variant of Homo sapiens IgE peptide
ADGAGCFINKQMADLELCPARAAEA

SEQ ID NO: 223 - protein, 25 aa, Artificial Sequence
  Artificial variant of Homo sapiens IgE peptide
ADGAGCFINRQLADMELCSRGAAEA

SEQ ID NO: 224 - protein, 25 aa, Artificial Sequence
  Artificial variant of Homo sapiens IgE peptide
ADGAECFINRQMADLELCGREAAEA

SEQ ID NO: 225 - protein, 25 aa, Artificial Sequence
  Artificial variant of Homo sapiens IgE peptide
ADGAGCFISPQLADWKRCMREAAEA

SEQ ID NO: 226 - protein, 25 aa, Artificial Sequence
  Artificial variant of Homo sapiens IgE peptide
ADGAGCSIHTQMADWERCLREGAEA

SEQ ID NO: 227 - protein, 25 aa, Artificial Sequence
  Artificial variant of Homo sapiens IgE peptide
ADGAGCSIHRQMADWERCLREGAEA

SEQ ID NO: 228 - protein, 16 aa, Artificial Sequence
  Artificial variant of Homo sapiens IgE peptide
CSSCDGGGHKPPTIQC

SEQ ID NO: 229 - protein, 20 aa, Artificial Sequence
  Artificial variant of Homo sapiens IgE peptide
CLQSSCDGGGHFPPTIQLLC

SEQ ID NO: 230 - protein, 15 aa, Artificial Sequence
  Artificial variant of Homo sapiens IgE peptide
APCWPGSRDCRTLAG

SEQ ID NO: 231 - protein, 16 aa, Artificial Sequence
  Artificial variant of Homo sapiens IgE peptide
ACPEWPGSRDRCTLAG

SEQ ID NO: 232 - protein, 17 aa, Artificial Sequence
  Artificial variant of Homo sapiens IgE peptide
CATPEWPGSRDKRTLCG

SEQ ID NO: 233 - protein, 16 aa, Artificial Sequence
  Artificial variant of Homo sapiens IgE peptide
CATPEWPGSRDKRTCG

SEQ ID NO: 234 - protein, 13 aa, Artificial Sequence
  Artificial variant of Homo sapiens IgE peptide
TPCWPGSRDKRCG

SEQ ID NO: 235 - protein, 19 aa, Artificial Sequence
  Artificial variant of Homo sapiens IgE peptide
CSRPSPFDLFIRKSPTITC

SEQ ID NO: 236 - protein, 18 aa, Artificial Sequence
  Artificial variant of Homo sapiens IgE peptide
CSRPSPFDLFIRKSPTIC

SEQ ID NO: 237 - protein, 17 aa, Artificial Sequence
  Artificial variant of Homo sapiens IgE peptide
CSRPSPFDLFIRKSPTC

SEQ ID NO: 238 - protein, 16 aa, Artificial Sequence
  Artificial variant of Homo sapiens IgE peptide
CSRPSPFDLFIRKSPC

SEQ ID NO: 239 - protein, 15 aa, Artificial Sequence
  Artificial variant of Homo sapiens IgE peptide
CRPSPFDLFIRKSPC

SEQ ID NO: 240 - protein, 16 aa, Artificial Sequence
  Artificial variant of Homo sapiens IgE peptide
CRPSPFDLFIRKSPTC

SEQ ID NO: 241 - protein, 17 aa, Artificial Sequence
  Artificial variant of Homo sapiens IgE peptide
CRPSPFDLFIRKSPTIC

SEQ ID NO: 242 - protein, 18 aa, Artificial Sequence
  Artificial variant of Homo sapiens IgE peptide
CRPSPFDLFIRKSPTITC

SEQ ID NO: 243 - protein, 17 aa, Artificial Sequence
  Artificial variant of Homo sapiens IgE peptide
CPSPFDLFIRKSPTITC

SEQ ID NO: 244 - protein, 16 aa, Artificial Sequence
  Artificial variant of Homo sapiens IgE peptide
CPSPFDLFIRKSPTIC

SEQ ID NO: 245 - protein, 15 aa, Artificial Sequence
  Artificial variant of Homo sapiens IgE peptide
CPSPFDLFIRKSPTC

SEQ ID NO: 246 - protein, 14 aa, Artificial Sequence
  Artificial variant of Homo sapiens IgE peptide
CPSPFDLFIRKSPC

SEQ ID NO: 247 - protein, 20 aa, Artificial Sequence
  Artificial variant of Homo sapiens IgE peptide
CYAFATPEWPGSRDKRTLAC

SEQ ID NO: 248 - protein, 19 aa, Artificial Sequence
  Artificial variant of Homo sapiens IgE peptide
CYAFATPEWPGSRDKRTLC

SEQ ID NO: 249 - protein, 18 aa, Artificial Sequence
  Artificial variant of Homo sapiens IgE peptide
CYAFATPEWPGSRDKRTC

SEQ ID NO: 250 - protein, 17 aa, Artificial Sequence
  Artificial variant of Homo sapiens IgE peptide
CYAFATPEWPGSRDKRC

SEQ ID NO: 251 - protein, 16 aa, Artificial Sequence
  Artificial variant of Homo sapiens IgE peptide
CAFATPEWPGSRDKRC

SEQ ID NO: 252 - protein, 17 aa, Artificial Sequence
  Artificial variant of Homo sapiens IgE peptide
CAFATPEWPGSRDKRTC

SEQ ID NO: 253 - protein, 18 aa, Artificial Sequence
  Artificial variant of Homo sapiens IgE peptide
CAFATPEWPGSRDKRTLC

SEQ ID NO: 254 - protein, 19 aa, Artificial Sequence
  Artificial variant of Homo sapiens IgE peptide
CAFATPEWPGSRDKRTLAC

SEQ ID NO: 255 - protein, 18 aa, Artificial Sequence
  Artificial variant of Homo sapiens IgE peptide
CFATPEWPGSRDKRTLAC

SEQ ID NO: 256 - protein, 17 aa, Artificial Sequence
  Artificial variant of Homo sapiens IgE peptide
CFATPEWPGSRDKRTLC

SEQ ID NO: 257 - protein, 16 aa, Artificial Sequence
  Artificial variant of Homo sapiens IgE peptide
CFATPEWPGSRDKRTC

SEQ ID NO: 258 - protein, 15 aa, Artificial Sequence
  Artificial variant of Homo sapiens IgE peptide
CFATPEWPGSRDKRC

SEQ ID NO: 259 - protein, 17 aa, Artificial Sequence
  Artificial variant of Homo sapiens IgE peptide
CTWSRASGKPVNHSTRC

SEQ ID NO: 260 - protein, 16 aa, Artificial Sequence
  Artificial variant of Homo sapiens IgE peptide
CTWSRASGKPVNHSTC

SEQ ID NO: 261 - protein, 15 aa, Artificial Sequence
  Artificial variant of Homo sapiens IgE peptide
CTWSRASGKPVNHSC

SEQ ID NO: 262 - protein, 14 aa, Artificial Sequence
  Artificial variant of Homo sapiens IgE peptide
CTWSRASGKPVNHC

SEQ ID NO: 263 - protein, 13 aa, Artificial Sequence
  Artificial variant of Homo sapiens IgE peptide
CWSRASGKPVNHC

SEQ ID NO: 264 - protein, 14 aa, Artificial Sequence
  Artificial variant of Homo sapiens IgE peptide
CWSRASGKPVNHSC

SEQ ID NO: 265 - protein, 15 aa, Artificial Sequence
  Artificial variant of Homo sapiens IgE peptide
CWSRASGKPVNHSTC

SEQ ID NO: 266 - protein, 16 aa, Artificial Sequence
  Artificial variant of Homo sapiens IgE peptide
CWSRASGKPVNHSTRC

SEQ ID NO: 267 - protein, 15 aa, Artificial Sequence
  Artificial variant of Homo sapiens IgE peptide
CSRASGKPVNHSTRC

SEQ ID NO: 268 - protein, 14 aa, Artificial Sequence
  Artificial variant of Homo sapiens IgE peptide
CSRASGKPVNHSTC

SEQ ID NO: 269 - protein, 13 aa, Artificial Sequence
  Artificial variant of Homo sapiens IgE peptide
CSRASGKPVNHSC

SEQ ID NO: 270 - protein, 12 aa, Artificial Sequence
  Artificial variant of Homo sapiens IgE peptide
CSRASGKPVNHC

SEQ ID NO: 271 - protein, 17 aa, Artificial Sequence
  Artificial variant of Homo sapiens IgE peptide
CQWLHNEVQLPDARHSC

SEQ ID NO: 272 - protein, 16 aa, Artificial Sequence
  Artificial variant of Homo sapiens IgE peptide
CQWLHNEVQLPDARHC

SEQ ID NO: 273 - protein, 15 aa, Artificial Sequence
  Artificial variant of Homo sapiens IgE peptide
CQWLHNEVQLPDARC

SEQ ID NO: 274 - protein, 14 aa, Artificial Sequence
  Artificial variant of Homo sapiens IgE peptide
CQWLHNEVQLPDAC

SEQ ID NO: 275 - protein, 13 aa, Artificial Sequence
  Artificial variant of Homo sapiens IgE peptide
CWLHNEVQLPDAC

SEQ ID NO: 276 - protein, 14 aa, Artificial Sequence
  Artificial variant of Homo sapiens IgE peptide
CWLHNEVQLPDARC

SEQ ID NO: 277 - protein, 15 aa, Artificial Sequence
  Artificial variant of Homo sapiens IgE peptide
CWLHNEVQLPDARHC

SEQ ID NO: 278 - protein, 16 aa, Artificial Sequence
  Artificial variant of Homo sapiens IgE peptide
CWLHNEVQLPDARHSC

SEQ ID NO: 279 - protein, 15 aa, Artificial Sequence
  Artificial variant of Homo sapiens IgE peptide
CLHNEVQLPDARHSC

SEQ ID NO: 280 - protein, 14 aa, Artificial Sequence
  Artificial variant of Homo sapiens IgE peptide
CLHNEVQLPDARHC

SEQ ID NO: 281 - protein, 13 aa, Artificial Sequence
  Artificial variant of Homo sapiens IgE peptide
CLHNEVQLPDARC

SEQ ID NO: 282 - protein, 12 aa, Artificial Sequence
  Artificial variant of Homo sapiens IgE peptide
CLHNEVQLPDAC

SEQ ID NO: 283 - protein, 17 aa, Artificial Sequence
  Artificial variant of Homo sapiens IgE peptide
CPSPFDLFIRKSPCGSK

SEQ ID NO: 284 - protein, 18 aa, Artificial Sequence
  Artificial variant of Homo sapiens IgE peptide
CPSPFDLFIRKSPTCGSK

SEQ ID NO: 285 - protein, 20 aa, Artificial Sequence
  Artificial variant of Homo sapiens IgE peptide
FAGCSRASGKPVNHCGAAEG

SEQ ID NO: 286 - protein, 21 aa, Artificial Sequence
  Artificial variant of Homo sapiens IgE peptide
FAGCSRASGKPVNHSCGAAEG

SEQ ID NO: 287 - protein, 22 aa, Artificial Sequence
  Artificial variant of Homo sapiens IgE peptide
FAGCSRASGKPVNHSTCGAAEG

SEQ ID NO: 288 - protein, 23 aa, Artificial Sequence
  Artificial variant of Homo sapiens IgE peptide
FAGCSRASGKPVNHSTRCGAAEG

SEQ ID NO: 289 - protein, 15 aa, Artificial Sequence
  Artificial variant of Homo sapiens IgE peptide
CSRASGKPVNHCGSK

SEQ ID NO: 290 - protein, 16 aa, Artificial Sequence
  Artificial variant of Homo sapiens IgE peptide
CSRASGKPVNHSCGSK

SEQ ID NO: 291 - protein, 17 aa, Artificial Sequence
  Artificial variant of Homo sapiens IgE peptide
CSRASGKPVNHSTCGSK

SEQ ID NO: 292 - protein, 23 aa, Artificial Sequence
  Artificial variant of Homo sapiens IgE peptide
FAGCFATPEWPGSRDKRCGAAEG

SEQ ID NO: 293 - protein, 24 aa, Artificial Sequence
  Artificial variant of Homo sapiens IgE peptide
FAGCFATPEWPGSRDKRTCGAAEG

SEQ ID NO: 294 - protein, 25 aa, Artificial Sequence
  Artificial variant of Homo sapiens IgE peptide
FAGCFATPEWPGSRDKRTLCGAAEG

SEQ ID NO: 295 - protein, 26 aa, Artificial Sequence
  Artificial variant of Homo sapiens IgE peptide
FAGCFATPEWPGSRDKRTLACGAAEG

SEQ ID NO: 296 - protein, 15 aa, Artificial Sequence
  Artificial variant of Homo sapiens IgE peptide
CPEWPGSRDKRCGSK

SEQ ID NO: 297 - protein, 13 aa, Artificial Sequence
  Artificial variant of Homo sapiens IgE peptide
CWPGSRDKRCGSK

SEQ ID NO: 298 - protein, 17 aa, Artificial Sequence
  Artificial variant of Homo sapiens IgE peptide
CPEWPGSRDKRCGAAEG

SEQ ID NO: 299 - protein, 20 aa, Artificial Sequence
  Artificial variant of Homo sapiens IgE peptide
FAGCLHNEVQLPDACGAAEG

SEQ ID NO: 300 - protein, 21 aa, Artificial Sequence
  Artificial variant of Homo sapiens IgE peptide
FAGCLHNEVQLPDARCGAAEG

SEQ ID NO: 301 - protein, 22 aa, Artificial Sequence
  Artificial variant of Homo sapiens IgE peptide
FAGCLHNEVQLPDARHCGAAEG

SEQ ID NO: 302 - protein, 23 aa, Artificial Sequence
  Artificial variant of Homo sapiens IgE peptide
FAGCLHNEVQLPDARHSCGAAEG

SEQ ID NO: 303 - protein, 20 aa, Artificial Sequence
  Artificial variant of Homo sapiens IgE peptide
FAGCLHNEVQLPDASGAAEG

SEQ ID NO: 304 - protein, 14 aa, Artificial Sequence
  Artificial variant of Homo sapiens IgE peptide
CPEWPGSRDRCGSK

SEQ ID NO: 305 - protein, 13 aa, Artificial Sequence
  Artificial variant of Homo sapiens IgE peptide
CWPGSRDRRCGSK

SEQ ID NO: 306 - protein, 20 aa, Artificial Sequence
  Artificial variant of Homo sapiens IgE peptide
CDSNPRGVSAADSNPRGVSC

SEQ ID NO: 307 - protein, 15 aa, Artificial Sequence
  Artificial variant of Homo sapiens IgE peptide
CLVVDLAPSKGTVNC

SEQ ID NO: 308 - protein, 9 aa, Artificial Sequence
  Artificial variant of Homo sapiens IgE peptide
CKQRNGTLC

SEQ ID NO: 309 - protein, 13 aa, Artificial Sequence
  Artificial variant of Homo sapiens IgE peptide
CEEKQRNGTLTVC

SEQ ID NO: 310 - protein, 8 aa, Artificial Sequence
  Artificial variant of Homo sapiens IgE peptide
CHPHLPRC

SEQ ID NO: 311 - protein, 10 aa, Artificial Sequence
  Artificial variant of Homo sapiens IgE peptide
CTHPHLPRAC

SEQ ID NO: 312 - protein, 12 aa, Artificial Sequence
  Artificial variant of Homo sapiens IgE peptide
CVTHPHLPRALC

SEQ ID NO: 313 - protein, 14 aa, Artificial Sequence
  Artificial variant of Homo sapiens IgE peptide
CRVTHPHLPRALMC

SEQ ID NO: 314 - protein, 16 aa, Artificial Sequence
  Artificial variant of Homo sapiens IgE peptide
CXRVTHPHLPRALMRC

SEQ ID NO: 315 - protein, 18 aa, Artificial Sequence
  Artificial variant of Homo sapiens IgE peptide
CQXRVTHPHLPRALMRSC

SEQ ID NO: 316 - protein, 20 aa, Artificial Sequence
  Artificial variant of Homo sapiens IgE peptide
CYQXRVTHPHLPRALMRSTC

SEQ ID NO: 317 - protein, 12 aa, Artificial Sequence
  Artificial variant of Homo sapiens IgE peptide
CPEWPGSRDKRC

SEQ ID NO: 318 - protein, 9 aa, Artificial Sequence
  Artificial variant of Homo sapiens IgE peptide
CRQRNGTLC

SEQ ID NO: 319 - protein, 13 aa, Artificial Sequence
  Artificial variant of Homo sapiens IgE peptide
CEERQRNGTLTVC

SEQ ID NO: 320 - protein, 16 aa, Artificial Sequence
  Artificial variant of Homo sapiens IgE peptide
CMRVTHPHLPRALMRC

SEQ ID NO: 321 - protein, 18 aa, Artificial Sequence
  Artificial variant of Homo sapiens IgE peptide
CQMRVTHPHLPRALMRSC

SEQ ID NO: 322 - protein, 20 aa, Artificial Sequence
  Artificial variant of Homo sapiens IgE peptide
CYQMRVTHPHLPRALMRSTC

SEQ ID NO: 323 - protein, 16 aa, Artificial Sequence
  Artificial variant of Homo sapiens IgE peptide
ACPEWPGSRDRCTLAG

SEQ ID NO: 324 - protein, 14 aa, Artificial Sequence
  Artificial variant of Homo sapiens IgE peptide
GGCLEDGQVMDVDC

SEQ ID NO: 325 - protein, 13 aa, Artificial Sequence
  Artificial variant of Homo sapiens IgE peptide
CLEDGQVMDCGSK

SEQ ID NO: 326 - protein, 16 aa, Artificial Sequence
  Artificial variant of Homo sapiens IgE peptide
CLEDGQVMDVDLCGSK

SEQ ID NO: 327 - protein, 22 aa, Artificial Sequence
  Artificial variant of Homo sapiens IgE peptide
CLEDGQVMDVDLCPREAAEGDK

SEQ ID NO: 328 - protein, 20 aa, Artificial Sequence
  Artificial variant of Homo sapiens IgE peptide
CLEDGQVMDVDLCGGSSGGK